(19) United States
(12) Patent Application Publication
(10) Pub. No.: US 2025/0381419 A1
(43) Pub. Date: Dec. 18, 2025

(54) SIMULTANEOUS INTENSITY AND ENERGY MODULATION AND COMPENSATION IN RADIOTHERAPY, METHODS OF RADIOTHERAPY, AND SYSTEMS OF RADIOTHERAPY

(71) Applicants: Shuang ZHOU, Atlanta, GA (US); Tiezhi ZHANG, Atlanta, GA (US); Steven BIEGALSKI, Atlanta, GA (US); Cristina OANCEA, Atlanta, GA (US); Minglei KANG, Atlanta, GA (US); Wei LIU, Atlanta, GA (US); Charles SIMONE, II, Atlanta, GA (US); Niklas WAHLS, Atlanta, GA (US); Emory University, Atlanta, GA (US)

(72) Inventors: Shuang Zhou, St Louis, MO (US); Tiezhi Zhang, St Louis, MO (US); Steven Biegalski, Atlanta, GA (US); Cristina Oancea, Atlanta, GA (US); Minglei Kang, Atlanta, GA (US); Wei Liu, Atlanta, GA (US); Charles Simone, III, Atlanta, GA (US); Niklas Wahls, Atlanta, GA (US); Liyong Lin, Atlanta, GA (US); Ruirui Liu, Atlanta, GA (US); Nathan Harrison, Atlanta, GA (US); Jeffery Bradkey, Atlanta, GA (US); Kristin Higgins, Atlanta, GA (US); William Dynan, Atlanta, GA (US); Jun Zhou, Atlanta, GA (US); Xiaofeng Yang, Atlanta, GA (US); Serdar Charyyev, Atlanta, GA (US); Alexander Stanforth, Atlanta, GA (US)

(21) Appl. No.: 18/879,054

(22) PCT Filed: Jun. 27, 2023
(86) PCT No.: PCT/US2023/069160

§ 371 Date: Dec. 26, 2024

Related U.S. Application Data
(60) Provisional application No. 63/355,750, filed on Jun. 27, 2022.

(51) Int. Cl. A61N 5/10

(52) U.S. Cl. CPC A61N 5/1077; A61N 5/1031; A61N 2005/1087; A61N 2005/1095

(57) ABSTRACT

The present disclosure provides for systems and methods for designing patient-specific sparse passive filters, for example compensation and modulation components, for simultaneous intensity and energy modulation in energetic entity or particle (e.g., proton) therapy, radiation therapy methods and systems, method for treating cancer in a patient or animal subject, method of optimizing an administration plan in particle FLASH radiotherapy or non-FLASH radiotherapy, configuration of the device or system to effectively place the patient-specific sparse passive filter, and the like.

### CLAIM OF PRIORITY TO RELATED APPLICATION

This application claims priority to co-pending U.S. provisional application entitled “FEASIBILITY OF PROTON SBRT FLASH TREATMENT WITH DOSE, DOSE RATE, AND LET OPTIMIZATION USING PATIENT SPECIFIC RIDGE FILTER” having Ser. No. 63/355,750, filed on Jun. 27, 2022, which is entirely incorporated herein by reference.

This application also claims priority to co-pending U.S. provisional application entitled “VALIDATION OF THE QUANTUM PHYSICS PROCESSES UNDERLYING THE INTEGRATED PHYSICAL OPTIMIZATION OF PROTON FLASH RADIOTHERAPY” having Ser. No. 63/433,202, filed on Dec. 16, 2022, which is entirely incorporated herein by reference.

This application also claims priority to co-pending U.S. provisional application entitled “QUANTUM PHYSICS SOLUTION TO THE INTEGRATED OPTIMIZATION OF DOSE, DOSE RATE, AND LET FOR PROTON FLASH THERAPY USING A DISTRIBUTED PARALLEL COMPUTING FRAMEWORK” having Ser. No. 63/446,479 filed on Feb. 17, 2023, which is entirely incorporated herein by reference.

### STATEMENT REGARDING FEDERALLY SPONSORED RESEARCH OR DEVELOPMENT

This invention was made with government support under contract number 75N91022C00055 awarded by the U.S. Department of Health and Human Services National Institutes of Health. The government has certain rights in this invention.

### BACKGROUND

Although stereotactic body radiation therapy (SBRT) provides excellent local tumor control, it poses unacceptable risks in certain subsets of patients. Stereotactic body proton therapy (SBPT) represents an advancement over SBRT, as it uses fewer beams and delivers much of the dose in a patient-specific spread-out Bragg Peak (SOBP), sparing proximal and especially distal organs at risk (OARs). Even with SBPT, there is necessarily some treatment margin, which may impact OARs and thus limit clinical applicability.

Proton FLASH radiotherapy is a new treatment modality that uses ultra-high dose rates (UHDR) and has the potential to provide further sparing of OARs beyond that offered by conventional SBPT. The current generation of proton therapy machines is, in many cases, capable of achieving FLASH dose rates (e.g., 40-800 Gy/second). In its technically simplest implementation, irradiation is performed using a high-energy transmission beam. However, active energy modulation is currently impractical, given that characteristic energy modulation times (>500 milliseconds) exceed the total time allowed for FLASH delivery (250 milliseconds for a typical 10 Gy SBPT dose). Unfortunately, the use of the transmission beam sacrifices a major advantage of proton therapy: the ability to deliver dose in a SOBP. For small SBPT targets other than extremities, the increased spillover to serial OARs can more than offset FLASH sparing. Passive energy modulation is perhaps the most promising approach for conformal delivery of FLASH fields, but designing the filters necessary for passive energy modulation is difficult.

In addition to the UHDR sparing effect, proton therapy planning must consider linear energy transfer (LET), a quantity related to radiation quality that can have a large impact on biological effectiveness. Lack of LET optimization for conformal FLASH compromises both clinical outcomes and the ability to interpret preclinical studies, as extra biological dose (XBD) attributable to high LET at the distal edge of the Bragg peak potentially offsets FLASH sparing.

### SUMMARY

Embodiments of the present disclosure provide for systems and methods for designing patient-specific sparse passive filters, patient-specific sparse passive filters for simultaneous intensity and energy modulation in energetic entity or particle (e.g., proton) therapy, radiation therapy methods and systems, method for treating cancer in a patient, method of optimizing an administration plan in a particle (e.g., proton) FLASH radiotherapy or non-FLASH radiotherapy, configuration of the device or system to effectively place the patient-specific sparse passive filter, and the like.

The present disclosure provides for radiation therapy methods, comprising: receiving a beam of particles; directing the beam of particles to a patient specific sparse passive filter to form an adjusted beam of particles, wherein the patient specific sparse passive filter is configured to modulate the beam of particles, wherein the patient specific sparse passive filter is formed based on a simultaneous optimization of a dose of particles from the beam of particles, a dose-averaged dose rate (DADR) of particles from the beam of particles, and dose-averaged linear energy transfer (LET_{d}) of the particles from the beam of particles to target a target area of a patient and substantially spare organs at risk (OARs); and administering the adjusted beam of particles to the target area of the patient.

The present disclosure provides for methods for treating cancer in a patient, the method comprising administering to the patient at least one fraction of proton ultra-high dose rate radiotherapy (FLASH), wherein the fraction of the proton beam pass through a patient specific sparse passive filter prior to being administered to the patient, wherein the patient specific sparse passive filter, is formed based on a simultaneous optimization of a dose of protons from the beam of protons, a dose-averaged dose rate (DADR) of protons from the beam of protons, and dose-averaged linear energy transfer (LET_{d}) of the protons from the beam of protons to target a target area of a patient and substantially spare organs at risk (OARs).

The present disclosure provides for systems for radiation therapy, comprising: a particle source for a beam of particles; and a patient specific sparse passive filter, wherein the patient specific sparse passive filter is configured in the system to receive the beam of particles, wherein the patient specific sparse passive filter is configured to modify the beam of particles to form an adjusted beam of particles, wherein the patient specific sparse passive filter is formed based on a simultaneous optimization of a dose of particles from the beam of particles, a dose-averaged dose rate (DADR) of particles from the beam of particles, and dose-averaged linear energy transfer (LETd) of the particles from the beam of particles to target a target area of a patient and substantially spare organs at risk (OARs).

The present disclosure provides for methods of optimizing an administration plan in particle FLASH radiotherapy, comprising: simultaneously optimizing a dose of particles from the beam of particles, a dose-averaged dose rate (DADR) of particles from the beam of particles, and dose-averaged linear energy transfer (LETd) of the particles from a beam of particles to a clinical target volume (CTV), beam-specific planning target volumes (BSPTVs), and organs at risk (OARs), wherein the optimization includes iteratively adjusting a geometry of patient-specific sets of geometric modulating and compensating components for a patient specific sparse passive filter, and the weight of a particle beam, optionally the weight of a proton pencil beam spot map, wherein simultaneously optimizing the dose of particles from the beam of particles, the DADR of particles from the beam of particles, and the LETd of the particles from the beam of particles, wherein the simultaneously optimizing is designed to reduce the dose of particles from the beam of particles, the DADR of particles from the beam of particles, and the LETd of the particles from a beam of particles in the OARs as compared to intensity modulated particles therapy; selecting an optimized patient specific sparse passive filter and an optimized weight of a particle beam optionally a weight of a proton pencil beam spot map; and implementing particle FLASH radiotherapy using the optimized patient specific sparse passive filter and the optimized weight particle beam, optionally the weight of a proton pencil beam spot map.

The present disclosure provides for methods of designing a patient specific sparse passive filter, comprising: receiving a scan of a patient; determining an initial geometry of a sparse passive filter based at least in part on the scan; determining a dose influence matrix and an LET influence matrix; in parallel with determining the dose influence matrix and the LET influence matrix, simulating a plurality of geometry variations using a particle simulation; and optimizing output data from the particle simulation to determine an optimized geometry, the optimization being based at least in part on the dose influence matrix and the LET influence matrix.

The present disclosure provides for patient-specific sparse passive filters for simultaneous intensity and energy modulation in proton therapy, the patient-specific sparse passive filter designed by the process of: determining an initial geometry of a sparse passive filter based at least in part on a scan of a patient; determining a dose influence matrix and an LET influence matrix; simulating a plurality of geometry variations using a particle simulation; and optimizing output data from the particle simulation to determine an optimized geometry; the optimization being based at least in part on the dose influence matrix and the LET influence matrix.

The present disclosure provides for systems for designing a patient-specific sparse passive filter, comprising: at least one computing device comprising a processor and a memory; and machine-readable instructions stored in the memory that, when executed by the processor, cause the computing device to at least: receive a scan of a patient; determine an initial geometry of a sparse passive filter based at least in part on the scan; determine a dose influence matrix and an LET influence matrix; in parallel with determining the dose influence matrix and the LET influence matrix, simulate a plurality of geometry variations using a particle simulation; and optimize output data from the particle simulation to determine an optimized geometry; the optimization being based at least in part on the dose influence matrix and the LET influence matrix.

The present disclosure provides for radiation therapy devices, comprising: a particle source for a beam of particles; and a nozzle that receives the beam of particles, wherein the nozzle includes a filter recessed within the nozzle.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 and FIG. 2 exemplify a specific approach for solving the IPO-IMPT problem FIG. 1A illustrates the beam's eye view (BEV) for a specific field of the CTV (small orange points), proton spot map (blue triangles), and filter modulation or compensation component (e.g., pins/bars) locations (black X symbols) (spacing increased for visual clarity). The dot-dash line with double-ended arrows represents the distance between a particular spot and modulation or compensation component. FIG. 1B illustrates a 3D grid representing the voxelized patient CTV. Each spot (red arrow/circle) has a unique restricted influence grid (blue box) that is much smaller than the full CTV grid. FIG. 1C illustrates a side view of a set of modulation/compensation components (in this example, 5 pins, 5 bars), and 8 spots (colorized for visual clarity) showing the interjoining of spots and pins. The geometry of each component (such as the length of each pin or bar) is variable. The dot-dash line with double-ended arrows represents the distance between a particular spot and the modulation/compensation component. FIG. 1D illustrates a simulation parallelization scheme for one patient and Ne treatment fields. A red X in the bottom row represents a simulation that can be skipped due to the spot being far away from the modulation/compensation component (represented by the dot-dash line with double-ended arrows in panels A and C).

FIG. 2 illustrates the overall workflow for optimization of the filter components and proton spot maps for a clinical (patient) treatment plan. We term this optimization workflow “Simultaneous Intensity and Energy Modulation and Compensation” (hereafter SIEMAC).

FIG. 3 illustrates a design for a preclinical (minipig) study, also optimized with the SIEMAC workflow. The setup includes an anterior 250 MeV proton pencil beam and sets of variable length pins and bars that can be optimized to irradiate a 36 mm diameter sphere within the lung.

FIG. 4 illustrates an optimized clinical treatment plan result. FIGS. 4A and B: Maps of spot weights, bar lengths (i.e., sparse compensation), and pin lengths (i.e., sparse modulation) for a single treatment field (field A, gantry 40° from perpendicular) before and after SIEMAC optimization, respectively. C and D: Dose, dose rate, and LET distributions for an axial slice of a 3-field patient plan before and after SIEMAC optimization, respectively. Also shown are contours of the left lung (blue), right lung (purple), heart (red), CTV (yellow), and three BSPTVs (white). E: The 12 components of the objective function (Equation 3, below) as a function of iteration number. F: Dose (top), dose rate (middle), and LET (bottom) volume histograms for the CTV (yellow), left lung (blue), and heart (red) before (solid line) and after (dashed line) SIEMAC optimization. Dose is reported for the whole structures (CTV, lung, heart), while DADR and LETd are reported only for regions of interest termed evaluation structures.

FIG. 5 illustrates animal study results showing dose (a and f), dose rate (b and g), LET (c and h), XBD (DADR) (d and i), and XBD (LET) (e and j) (non-cumulative) distributions for the spherical target of the minipig for the design described in described in FIG. 3. The bottom row (f-j) shows results before (blue) and after (red) optimization was done to reduce the spread in dose, DADR, and LET_{d}.

FIG. 6 illustrates examples of energy compensation and modulation components such as pin, bars, stacks, pin-sets, bar-sets, and stack-sets.

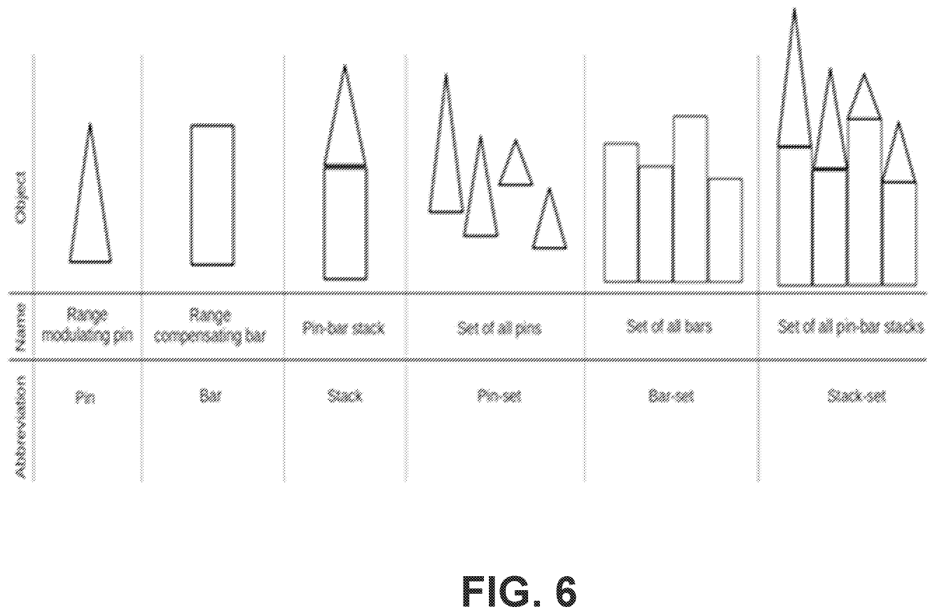

FIG. 7 illustrates the simulation showing restricted influence grid (RIG).

FIG. 8 illustrates a quality control setup for experimental measurements to validate that actual dose rates and LETs correspond to planned or predicted values. FIG. 8A illustrates the experimental design showing the ridge filter, 30 mm lucite range shifter, 80 mm block of additional lucite, and water phantom; (B) a photo of the setup for the dose and dose rate measurements done with the MLSIC detector; (C) a photo of the setup for the LET and timing measurements done with the two Timepix3 detectors.

FIG. 9 illustrates dose distributions for the one spot, as measured using the setup in FIG. 8. A: Axial slice at depth 50 mm (top) and sagittal slice at x=0 (bottom) from the simulation. B: Axial slice at depth 50 mm (top) and sagittal slice at x=0 (bottom) from experimental MLSIC data. C: Relative dose profile at depth 50 mm for MLSIC data (orange) and simulation (blue) (left) and relative depth dose curve for MLSIC data (orange) and simulation (blue).

FIG. 10 illustrates time-dependent instantaneous dose rate fluctuations for spot 1 measured with the MLSIC detector at a depth of 50 mm along the central axis of the spot for 7 nA (blue) and 50 nA (red).

FIG. 11 illustrates the LET measurements with the primary pixelated detector. FIG. 11A: Three representative LET distributions with different acquisition times (500, 200, and 100 μs, respectively) with Gaussian fits used to find the peak position. FIG. 11B: LET peak position vs acquisition time using results from (11A). FIG. 11C: Two representative LET distributions with different detector angles (0° and 45°, respectively) with Gaussian fits used to find the peak position. FIG. 11D: LET peak position vs detector angle using results from (11C).

FIG. 12 illustrates the corrected experimental and simulated LET distributions for five locations. FIG. 12A illustrates a sagittal view of dose distribution with dashed lines showing the spherical target, the central axis, and the five locations where the LET was measured. FIG. 12B-F illustrates LET distributions for simulation (blue dashed line) and Advapix Timepix3 data (solid orange line) at 30 mm depth on axis (12B), 60 mm depth at lateral margin (12C), 85 mm depth on axis (12D), 90 mm depth on axis (12E), and 95 mm depth on axis (12F).

FIG. 13A illustrates a sagittal slice at the mid-plane (x=0) of absolute dose from the Monte Carlo simulation; the dashed lines represent the spherical target, the central axis, and three representative depths which are shown in more detail in (13C)-(13E). 13B illustrates the depth dose along the central axis (x=y=0) for MatriXX PT data (orange) and simulation (blue); the dashed lines represent the three depths which are shown in more detail in (13C)-(13E). 13C-E illustrates the absolute dose distributions at depths of 90 mm, 60 mm, and 28 mm, respectively, for data and simulations; in (13C), an additional simulation result is shown at depth 91 mm to show that the disagreement is within 1 mm.

FIG. 14A illustrates a simplified model involving a uniform water phantom with a 50 mm diameter spherical target and 10 mm margin at a depth of 60 mm. A sparse passive filter is used to create the desired proton energy fluence. The green “+” symbols highlight the proximal, distal, and lateral margins, which are regions of particular importance. FIG. 14B illustrates a Monte Carlo simulation of (from right to left) the sparse passive filter, a 30 mm range shifter, the 80 mm PMMA block, and water phantom. Also shown by blue lines are 250 MeV protons traveling from right to left along with secondary particles shown in red and green.

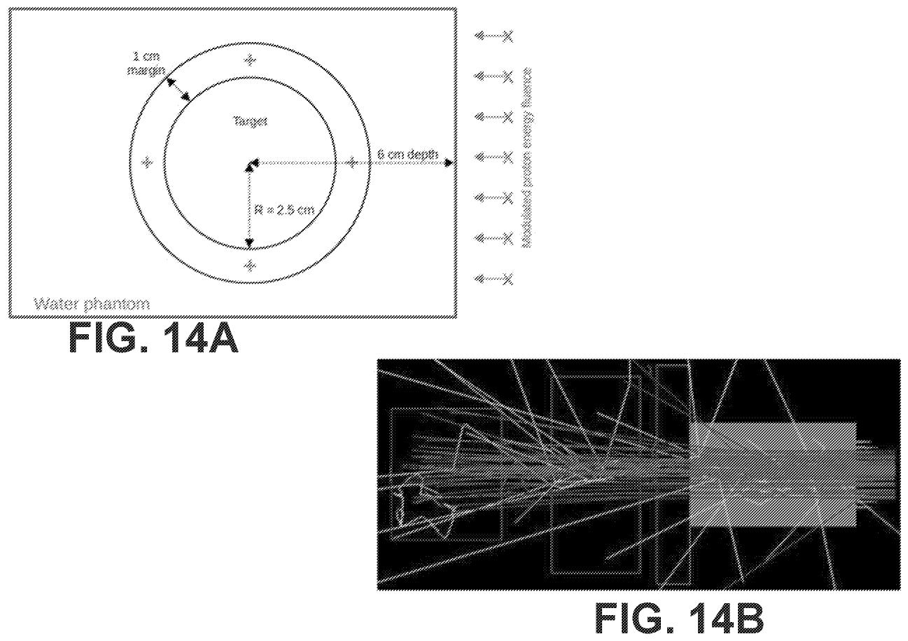

FIG. 15A illustrates the outer (downstream) part of the nozzle (a piece of hardware through which protons flow from the accelerator into the treatment room and thence into the patient). FIG. 15B illustrates the inner (upstream) part of the nozzle where the filter assembly (including modulation/compensation components) is mounted. The placement of the filter assembly in the inner (upstream) part of the nozzle provides roughly 40% higher dose rate than placement on the outer (downstream) part. This is because it allows the patient to be closer to the nozzle. FIG. 15C illustrates the photos and CAD images of the components of the sparse passive filter, which include a reusable uniform base, a compensator, and pins. FIG. 15D illustrates a block diagram illustrating the sparse passive filter positioned on the nozzle of the proton FLASH radiotherapy system or device, whereas FIG. 15E illustrates a block diagram illustrating the sparse passive filter positioned within the nozzle.

FIG. 16A illustrates the instant dose rate for a single spot with 50 nA nozzle current. FIG. 16B illustrates the average dose rate including all spots (total dose divided by total irradiation time) with 50 nA nozzle current.

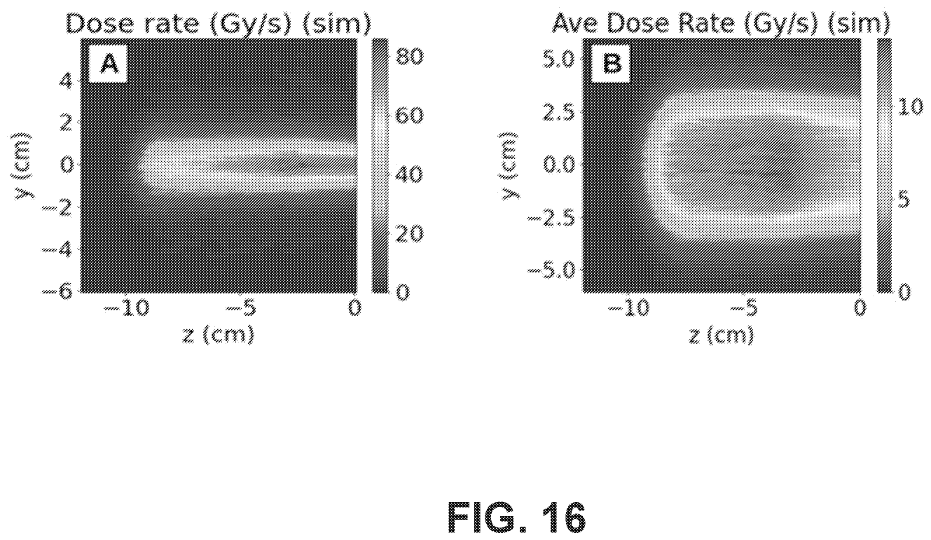

FIG. 17 illustrates timing with secondary detector (Minipix Timepix3) at 250 MeV and 10 nA. Four repetitions were done to demonstrate reproducibility.

### DEFINITIONS

BEV: “Beam's eye view.” A notional view along the beam axis often used in quality assurance and planning for external beam radiotherapy.

BSPTV: “Beam-specific planning target volume.” The BSPTV is created by adding geometric margins to the clinical target volume. BSPTV allows for individualizing the magnitude of each margin for each treatment field.

CTV: “Clinical Target Volume.” The tissue volume that contains the gross tumor volume and subclinical microscopic malignant lesions.

DADR: “Dose averaged dose rate.” The dose-weighted mean of the dose rates of all scanning proton spots averaged over the duration of the irradiation.

FLASH: A radiotherapy technique for photon and proton treatments, using dose rates that are much higher than in conventional radiotherapy, with the aim of sparing normal tissue while maintaining anti-tumor efficacy.

IPO-IMPT: “Integrated physical optimization-IMPT.” A framework which can selectively optimize radiation parameters (i.e., reduce the LET_{d} or increase the DADR) to OARs for sparing the potential toxicity while keeping good dose coverage constraints to target.

IMPT: “Intensity modulated proton therapy.” Currently, the most precise type of proton delivery. More closely conforms to the tumor while avoiding OARs. Allows for dose modulation along the beam axis as well as lateral, in-field dose modulation.

LET: “Linear energy transfer.” An indicator of radiation quality of ion beams. LET varies inversely with velocity (kinetic energy) of the ions.

LET_{d}: “Dose averaged LET.” Frequently used as a representative quantity for the biological effectiveness of a radiation field. Considers the stopping power of each individual particle, weighted by its contribution to the local dose. A single-valued metric to describe the particle system.

MFO: “Multi-field optimization.” The simultaneous spot optimization of all fields, for example, successive irradiations at different beam angles.

OARs: “Organs at risk.” Healthy tissues and organs which are located near the target of the radiotherapy. Damage to the OARs from irradiation may require changes to the radiotherapy treatment plan. Examples in the context of lung cancer include the heart, great vessels, and esophagus.

SBPT: “Stereotactic body proton therapy.” A type of radiation therapy which uses fewer beams than SBRT and delivers much of the dose in patient-specific SOBPs, sparing proximal and especially distal OARs.

SBRT: “Stereotactic body radiotherapy.” A type of radiation therapy that uses many beams of energy carefully targeted to tumors. SBRT is differentiated from other radiation therapy because it is delivered in 5 or fewer fractions (treatment sessions) each with a comparatively high dose, typically 8 Gy or more per fraction.

SFO: “Single-field optimization.” Each beam is optimized individually to deliver the prescribed dose to the target.

SIEMAC: “Simultaneous intensity and energy modulation and compensation.” A new inverse optimization approach described herein.

SOBP: “Spread out Bragg Peak.” A Bragg peak is a peak of dose at the end of the proton track where the kinetic energy falls to zero. A Spread Out Bragg Peak is the sum of multiple individual Bragg peaks from beams of slightly different energies, carefully designed to deliver a plateau of dose within a cuboid, with near-zero dose on the distal side. The peak dose is not reached until deep in the tissue, allowing for treatment to conform to larger tumors and more specific 3D shapes.

Sparse passive filter: Filter from which some range modulation or range compensation geometric components, such as pins and bars, have been reduced in size, shortened, or omitted, in order to optimize dose rate and LET. In other aspects, the geometric components, such as pins and bars, can have increased size and/or length to optimize dose rate and LET.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of physics, material science, computer science, medical imaging, radiation biology and the like, which are within the skill of the art. Such techniques are explained fully in the literature.

In the following detailed description, for purposes of explanation and not limitation, exemplary, or representative, embodiments disclosing specific details are set forth in order to provide a thorough understanding of inventive principles and concepts. However, it will be apparent to one of ordinary skill in the art having the benefit of the present disclosure that other embodiments according to the present teachings that are not explicitly described or shown herein are within the scope of the appended claims. Moreover, descriptions of well-known apparatuses and methods may be omitted so as not to obscure the description of the exemplary embodiments. Such methods and apparatuses are clearly within the scope of the present teachings, as will be understood by those of skill in the art. It should also be understood that the word “example,” as used herein, is intended to be non-exclusionary and non-limiting in nature.

### Discussion

Radiation therapy is a key therapeutic modality for treating cancer. A beam of energy can be delivered to a tumor to break chemical bonds, including those within the tumor cells' DNA. Radiation therapy offers the benefits of sub-millimeter precision while mostly sparing normal tissue, ultimately leading to death of tumor cells. Although stereotactic body radiation therapy (SBRT), which uses many beams of radiation to deliver extremely precise and intense doses of radiation, provides excellent local tumor control, it poses unacceptable risks in a subset of patients. For example, patients with central and ultra-central lung tumors are at a 15% risk of fatal hemorrhage based on impingement of the complex overlapping radiation fields on organs at risk (OARs), including uninvolved lung, heart, and esophagus. Stereotactic body proton therapy (SBPT) represents an advancement over SBRT as it uses fewer beams and delivers much of the dose in a patient-specific spread-out Bragg Peaks (SOBPs), sparing proximal and especially distal OARs. Even with SBPT, there is necessarily some treatment margin, which may impact OARs and thus limit clinical applicability.

The present disclosure provides for systems and methods for designing patient-specific sparse passive filters, patient-specific sparse passive filters for simultaneous intensity and energy modulation in energetic entity or particle (e.g., proton) therapy, radiation therapy methods and systems, methods for treating cancer in a patient, methods of optimizing an administration plan in a particle (e.g., proton) FLASH radiotherapy or non-FLASH radiotherapy, configurations of the device or system to effectively place the patient-specific sparse passive filter, and the like. Aspects of the present disclosure provide for systems and methods that combine a patient-specific sparse passive filter with a range compensator to achieve a single field-optimized (SFO) or multi-field-optimized (MFO), conformal dose distribution similar to the dose distribution obtained by conventional IMPT (intensity modulated proton therapy) or other energetic entity therapy.

Embodiments of the present disclosure provide for FLASH radiotherapy devices, systems, methods, constructs (e.g., sparse passive filter) that describe the administration of an energy using a suitable system or device for delivery the energy, for example, an electron linear accelerator, a proton source, or a source of ions heavier than protons. FLASH radiotherapy can be administered using, for example, high energy charged particles (e.g., protons, or ions that are heavier than protons (for example, helium, lithium, carbon, or neon atomic nuclei) or electrons). In an aspect, the energetic particles are protons, or ions that are heavier than protons (for example, helium, lithium, carbon, or neon atomic nuclei), or electrons. In this regard, the sparse passive filter can modulate (e.g., degrade) beams of energetic particles such as protons, ions that are heavier than protons, and electrons. In a particular aspect, the energetic particles are protons. In this regard, the sparse passive filter can modulate (e.g., degrade) beams of protons.

In addition, while much of the description describes FLASH radiotherapy, aspects of the present disclosure can be used in non-FLASH radiotherapy such as a lower dose rate (non-FLASH) radiotherapy where simultaneous modulation of dose, dose rate, and/or LET is desired.

In an effort to clearly describe features of the present disclosure, the present disclosure presents aspects using proton FLASH radiotherapy. The present disclosure is not limited to only using protons, as other energetic entities can be used such as ions that are heavier than protons (for example, helium, lithium, carbon, or neon atomic nuclei) or electrons. Also, the present disclosure is not limited to FLASH radiotherapy, and other non-FLASH therapy can be used with aspects of the present disclosure.

Aspects of the present disclosure address the technical problem of simultaneous optimization of dose, dose rate, and LET as Integrated Physical Optimization of Intensity Modulated Proton Therapy (IPO-IMPT) (or other energetic entities).

The present disclosure provides for an inverse optimization approach, termed Simultaneous Intensity and Energy Modulation and Compensation (SIEMAC), that can optimize dose, dose rate, and linear energy transfer (LET), simultaneously. In particular, SIEMAC can simultaneously optimize a dose of a particle from the beam of the particles, a dose-averaged dose rate (DADR) of the particle from the beam of the particles, and dose-averaged linear energy transfer (LET_{d}) of the particle from the beam of the particles administered to an area of a patient (e.g., human) with reduced effect on surrounding tissue and organs. This method includes iteratively optimizing the geometry of patient-specific sets of modulation and compensation components of a patient-specific sparse passive filter, such as range-compensating bars and range-modulating pins, and the weight (e.g., dose) of energetic entity, to deliver more desirable dose, dose rate (e.g., DADR), and LET distributions (e.g., LET_{d}) to the clinical target volume (CTV), beam-specific planning target volumes (BSPTVs), and organs at risk (OARs) when compared with more conventional techniques. For preclinical applications, SIEMAC reduces the spread of dose, dose rate (e.g., DADR), and LET distributions (e.g., LET_{d}) in OAR irradiations.

In a particular aspect, the present disclosure provides for an inverse optimization approach, termed Simultaneous Intensity and Energy Modulation and Compensation (SIEMAC), that can optimize dose, dose rate, and linear energy transfer (LET) simultaneously. In particular, SIEMAC can simultaneously optimize a dose of protons from the beam of protons, a dose-averaged dose rate (DADR) of protons from the beam of protons, and dose-averaged linear energy transfer (LET_{d}) of the protons from the beam of protons administered to an area of a patient (e.g., human) with a reduced effect on surrounding tissue and organs. This method includes iteratively optimizing the geometry of patient-specific sets of modulation and compensation components of a patient-specific sparse passive filter, such as range-compensating bars and range-modulating pins, and the weight (e.g., dose) of a proton pencil beam spot map, to deliver more desirable dose, dose rate (e.g., DADR), and LET distributions (e.g., LET_{d}) to the clinical target volume (CTV), beam-specific planning target volumes (BSPTVs), and organs at risk (OARs) when compared with more conventional techniques. For preclinical applications, SIEMAC reduces the spread of dose, dose rate (e.g., DADR), and LET distributions (e.g., LET_{d}) in OAR irradiations.

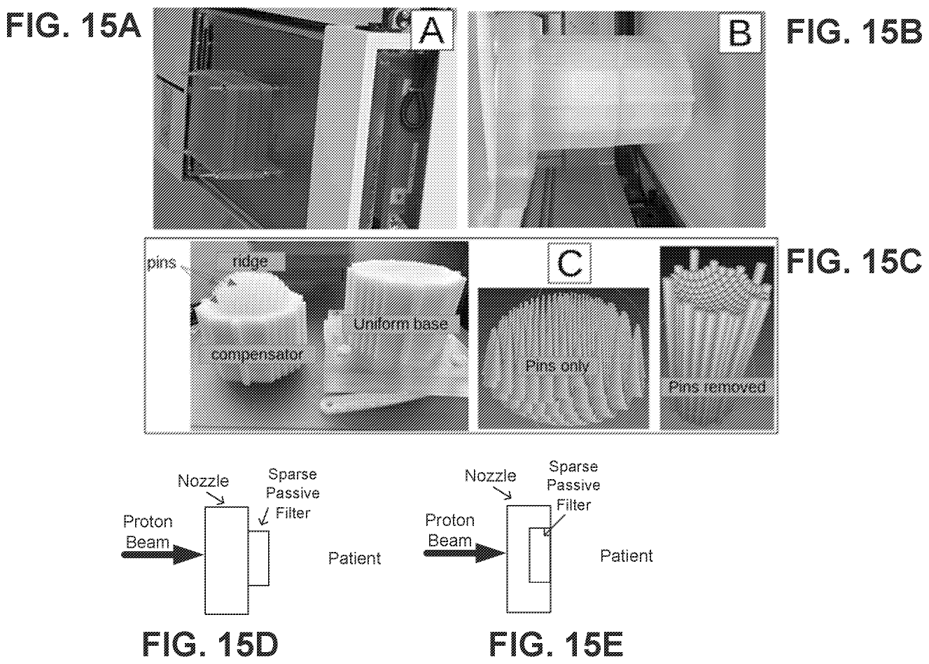

In general, the proton FLASH radiotherapy system or device include at least a proton source (e.g., FLASH irradiator and accelerator), a beam transport system, a patient specific sparse passive filter, and a range compensator, as well as other components that are part of a proton FLASH radiotherapy device or system. In an aspect, the patient specific sparse passive filter is positioned (e.g., recessed) within a nozzle of the system or device that is adjacent to a patient. Placement of all or part of the patient specific sparse passive filter assembly so that it is recessed within the nozzle can increase the dose rate by about 30% or more, about 35% or more or about 40% or more as compared to the patient specific sparse passive filter positioned outside (e.g., not recessed) of the nozzle, which allows the nozzle with the recessed patient specific sparse passive filter to be positioned closer to the patient while other parameters are equivalent between the proton FLASH radiotherapy device or system with the recessed and non-recessed patient specific sparse passive filter. In a particular aspect, the increased dose rate described above can be achieved if the length of patient specific sparse passive filter is about 20 cm and the source to iso-center distance is about 200 cm. The patient specific sparse passive filter can be placed as close as possible to the monitor unit chamber of the proton FLASH radiotherapy system, which monitors the location and energy of the scanning protons to modify incoming proton energies, i.e., dose rate and LET, according to SIEMAC, which modulates the incoming proton intensities and energies before exiting the proton FLASH radiotherapy system. The state of the art has the filter positioned outside of the nozzle. This improvement is because the patient to be closer to the nozzle. FIG. 15A illustrates the outer (downstream) part of the nozzle (a piece of hardware through which protons flow from the accelerator into the treatment room and thence into the patient). FIG. 15B illustrates the inner (upstream) part of the nozzle where the patient specific sparse passive filter assembly (including modulation/compensation components) is mounted. FIG. 15D illustrates a block diagram illustrating the sparse passive filter positioned on the nozzle of the proton FLASH radiotherapy system or device, whereas FIG. 15E illustrates a block diagram illustrating the sparse passive filter positioned so that it is recessed within the nozzle. While the recessed placement of the sparse passive filter within the nozzle is described in the context with proton FLASH radiotherapy systems or devices, other filters can benefit by positioning within nozzle and see an increased dose rate (e.g., about 30% or more, about 35% or more, or about 40% or more) and/or can be used in other particle FLASH radiotherapy and non-FLASH radiotherapy. Such mounting can be typically achieved within mm accuracy and can be validated by quality assurance (QA) before patient treatment to ensure the sparse design, manufacturing and mounting are within agreement of intended treatment planning.

Other types of FLASH radiotherapy systems and devices would include equivalent components specific for the particle. The proton source can be formed into a proton beam with a desired intensity, and energy, which is directed through the patient specific passive filter and the range compensator and ultimately into area volume within the patient's body (e.g., cancer (tumor)). Exemplary devices that may be used to administer FLASH radiation are described in, for example, U.S. Pat. No. 9,855,445, which is incorporated by reference and proton FLASH radiotherapy systems and device by VARIAN MEDICAL SYSTEMS.

Proton beam treatment has the advantage of being able to penetrate deeper into the tissue than electron beams. Furthermore, proton beams deposit the maximum of their energy at the end of their path (the Bragg peak), reducing harm to healthy tissue. FLASH proton beam treatment is delivered at higher dose rates than conventional proton beam treatment, which affect the biological response to radiation in a way that spares normal tissue while maintaining anti-tumor efficacy. FLASH proton radiotherapy may be administered using a passive beam scattering system (e.g., a single scattering system or double scattering system) or a dynamic spot scanning system. In an embodiment, the radiation therapy or treatment system used to deliver proton FLASH radiotherapy is a proton pencil beam scanning system.

The dose of the proton FLASH radiotherapy that can be administered to a patient (e.g., human) depends on the characteristics of the patient and the cancer being treated. In some embodiments, the dose of proton FLASH radiotherapy administered is about 1 to 100 Gy or about 1 Gy and 70 Gy. A dose of proton FLASH radiotherapy can be administered at a rate of about 40 Gy/sec to 300 Gy/sec or more. The dose of proton FLASH radiotherapy can be administered as fractionated doses, i.e., in a series of smaller doses over a period of time. Generally, conventional radiotherapy is fractionated into separate doses administered over days or weeks in order to achieve acceptable results. For example, in SBRT, the dose can be administered in 5 fractions. The dose is administered to an area of the patient (e.g., location of the tumor) and can be described as the clinical target volume (CTV).

The proton FLASH radiotherapy may be delivered in a pulsed manner, a continuous manner, or a quasi-continuous manner. In some embodiments, the proton FLASH radiotherapy can be administered in a pulsed manner with pulses at a frequency of about 100 Hz. In an aspect, the dose of proton FLASH radiotherapy can be delivered in a single pulse or can be delivered in a series of two or more pulses. Each pulse can have a duration of less than a second, several seconds, or several minutes. The interval between pulses may also last less than a second, several seconds, or several minutes. In some embodiments, each pulse in a series of pulses can have the same duration of different durations. In some embodiments, the intervals between each pulse in a series of pulses have the same duration or different durations. The dose and pulse parameters may be varied to optimize the desired result.

The purpose of the administration of the proton FLASH radiotherapy is to ablate or control the growth and progression of a cancer from a patient such as a human or other mammal (e.g., cat, dog, horse, cattle, and the like). The cancer can include thoracic cancer (e.g., lung cancer), head and neck cancer, brain cancer (e.g., glioblastoma), skin cancer, prostate cancer, pelvic cancer or liver cancer.

Although patient-specific sparse passive filters have clear advantages, designing such filters is challenging. Specifically, there is an unmet need to optimize sparse passive filter design and spot maps to maximize the sparing of organs at risk (OARs). Because dose of the proton beam, dose-averaged dose rate (DADR) of the proton beam, and dose-averaged linear energy transfer (LET_{d}) of the proton beam each influence the biological response, simultaneous optimization of all three factors is desirable, which can be achieved using embodiments of the present disclosure. In an aspect, the SIEMAC framework described herein achieves this goal. Using this framework, patient-specific sparse passive filters (from which some pins and/or have been shortened or omitted) can be developed which offer advantages over (non-optimized) regularly-spaced ridge filters (hereafter referred to as regular ridge filters) by increasing the volume that meets the FLASH dose rate threshold, thus maximizing FLASH sparing. In another aspect, using this framework, patient-specific sparse passive filters (from which some pins and/or bars have been lengthened are enlarged) can be developed which offer advantages over (non-optimized) regularly-spaced ridge filters (hereafter referred to as regular ridge filters) by increasing the volume that meets the FLASH dose rate threshold, thus maximizing FLASH sparing.

In general, a filter is a beam energy modulation device which is typically represented by a number of geometric components, such as pins and bars. In an aspect, the geometric components can have a variety of shapes (e.g., a polygonal cross section), combinations of shapes, and dimensions (e.g., 1 mm to 10s or 100s of mm) to achieve the desired goal of the sparse passive filter (e.g., calculated to allow a conformal dose distribution in the BSPTV). In a traditional filter, for example, each component, or pin, is a degrader of the proton beam with a length calculated to allow a conformal dose distribution in the BSPTV. Together, the pins combine to allow the formation of a spread-out Bragg Peaks (SOBP). The form of the SOBP in a region can be adjusted, such as altering a pin. The energy of protons passing through the different thicknesses of the sparse passive filter is modulated. Such a filter has a different amount of material and provides different energy modulation at different spot locations. Nevertheless, the design of that device leads to an increase in the dose within the planned target volume (BSPTV).

The patient specific compensation and modulation component of the present disclosure (also referred to as optimized patient specific sparse passive filter) is designed to simultaneously optimize dose, DADR and LET_{d} distributions. It can be made from a plastic material, such as polyethylene or Lucite, wax, or some other material. The patient specific sparse passive filter can include compensation and modulation geometric components. For example, the patient specific sparse passive filter can include compensation geometric components and modulation geometric components such as compensation bars and modulation pins. The patient specific sparse passive filter can include compensation and modulation geometric components in some areas and none in other areas and/or compensation and modulation geometric components are reduced in size and/or length (e.g., as compared to normal ridge filters), where the specific design depends upon the patient specific aspects of the cancer (e.g., tumor) being treated. The height of the geometric components (e.g., modulation pins and compensation bars) of the patient specific sparse passive filter can be about 1 to 100 mm. The cross-sectional width of the geometric components (e.g., modulation pins and compensation bars) can be about 1 to 25 mm or 1 to 5 mm. In a particular aspect, the modulation pins can have a triangular cross section and can have a conical three-dimensional shape while the compensation bars can have a rectangular cross section. It should be noted that the geometric components (e.g., compensation bars and modulation pins) can be referred to as a “ridge”. However, the compensation bars and modulation pins do not need to be in “ridge” format and can be in coarser resolution to achieve energy modulation and some sparsity at desired locations. Aspects of the present disclosure can be used to develop patient specific sparse passive filter based on desired values for dose, DADR, and LET_{d}.

The patient specific sparse passive filter is positioned on a range compensator. The particle beam (e.g., proton beam) is directed through the patient specific sparse passive filter assembly, that is, through the patient specific sparse passive filter and the range compensator and ultimately into a volume within the patient (e.g., tumor). Range compensators are hardware configured to finely adjust or reduce the range of the shifted ionizing radiation to account for the three-dimensional shape of the target tissue. A range compensator can further adjust the beam such that different portions of the proton beam penetrate the target at different depths. The compensator may be made from a plastic material, such as polyethylene or Lucite, wax, or some other material.

Aspects of the present disclosure can include patient specific sparse passive filter and optionally range compensators that can modulate the energy of the proton beam to achieve the desired dose, dose-averaged dose rate (DADR), and dose-averaged linear energy transfer (LET_{d}).

Having described some features of the present disclosure, additional features are now presented. As stated above, while aspects of the present disclosure are described in terms of “protons”, other particles can be used instead of protons. Also, while FLASH radiotherapy is described, aspects of the present disclosure are not limited to FLASH radiotherapy and can be implemented in non-FLASH therapy.

The present disclosure provides for methods of optimizing an administration plan to be used in proton FLASH radiotherapy. The optimized plan can be developed based on the patient and the disease state of the patient (e.g., tumor location, tumor size, and the like). The method includes simultaneously optimizing a dose of protons from the beam of protons, a dose-averaged dose rate (DADR) of protons from the beam of protons, and dose-averaged linear energy transfer (LET_{d}) of the protons from a beam of protons to a clinical target volume (CTV), beam-specific planning target volume (BSPTV), and organs at risk (OARs). The optimization includes iteratively adjusting a geometry of patient-specific sets of range compensating geometric components (e.g., bars) and range modulating geometric components (e.g., pins) for a patient specific sparse passive filter, and the weight of proton beam (e.g., a proton pencil beam spot map). Simultaneously optimizing the dose of protons from the beam of protons, the DADR of protons from the beam of protons, and the LET_{d} of the protons from the beam of protons reduces (e.g., by about 10%, by about 20%, by about 30%, by about 50%, by about 70%, by about 80%, by about 90%, or more) the dose of protons from the beam of protons, the DADR of protons from the beam of protons, and the LET_{d} of the protons from a beam of protons in the OARs as compared to intensity modulated proton therapy (which is the state-of-the art at this time). The method can include production of optimized patient specific sparse passive filters that can be used with proton beams, in particular optimized proton beams (e.g., a beam intensity used in the design of the optimized patient sparse passive filter). Additional details are provided in Examples 1-3.

The method also includes selecting an optimized patient specific sparse passive filter, and an optimized proton beam (e.g., an optimized weight of a proton pencil beam spot map) based on the foregoing step. As described herein, the present disclosure provides for making and using the optimized patient specific sparse passive filter. The method includes implementing proton FLASH radiotherapy plan by using the optimized patient specific sparse passive filter and optionally the optimized proton beam strength (e.g., optimized weight of a proton pencil beam spot map).

The present disclosure also provides for a radiation therapy method. The method can include receiving a beam of protons and directing the beam of protons to a patient specific sparse passive filter (e.g., as described and designed according to the present disclosure) to form an adjusted beam of protons. The patient specific sparse passive filter is configured to modulate the beam of protons. The patient specific sparse passive filter is formed based on a simultaneous optimization of a dose of protons from the beam of protons, a dose-averaged dose rate (DADR) of protons from the beam of protons, and dose-averaged linear energy transfer (LET_{d}) of the protons from the beam of protons to modulate the proton beam to optimally target a volume within a patient. In an aspect, the patient specific sparse passive filter (and optionally the intensity of the proton beam) are configured to substantially (e.g., reducing relative to a non-optimized patient specific sparse passive filter and optionally the non-optimized strength of the proton beam, where reducing can be about 10% less toxicity to OARs, about 20% less toxicity to OARs, about 40% less toxicity to OARs, about 60% less toxicity to OARs, about 80% less toxicity to the OARs, or about 90% less toxicity to the OARs) avoid organs at risk (OARs) while delivering a therapeutically effective amount of protons to the target volume (e.g., tumor). The method also includes administering the adjusted beam of protons to the target volume of the patient. In an aspect, the administration can include single field-optimized (SFO), or multi-field-optimized conformal dose distribution of the protons to the target area of the patient using the patient specific sparse passive filter.

In a particular aspect, the present disclosure provides for methods for treating cancer in a patient. The method can include administering to the patient at least one fraction (e.g., 1 to 5 fractions) of proton ultra-high dose rate radiotherapy (FLASH) (optionally optimized dose of the protons). At least one fraction of the proton beam passes through a patient specific sparse passive filter prior to being administered to the patient. The patient specific sparse passive filter can be formed based on a simultaneous optimization of a dose of protons from the beam of protons, a dose-averaged dose rate (DADR) of protons from the beam of protons, and dose-averaged linear energy transfer (LET_{d}) of the protons from the beam of protons directed at a target area of a patient. Also, the patient specific sparse passive filter can result in the proton beam substantially (defined above) reducing toxicity to organs at risk (OARs). The method also includes administering the adjusted beam of protons to the target area of the patient. In an aspect, the administration can include single field-optimized (SFO), or multi-field-optimized conformal dose distribution of the protons to the target area of the patient using the patient specific sparse passive filter.

The present disclosure also provides for systems for radiation therapy. In an aspect, the system can include a proton source for a beam of protons and a patient specific sparse passive filter (as optimized according to the present disclosure). The patient specific sparse passive filter is configured in the system to receive the beam of protons (optionally optimized dose of the protons). The patient specific sparse passive filter is configured to modify the beam of protons to form an adjusted beam of protons, where the patient specific sparse passive filter is formed based on a simultaneous optimization of a dose of protons from the beam of protons, a dose-averaged dose rate (DADR) of protons from the beam of protons, and dose-averaged linear energy transfer (LET_{d}) of the protons from the beam of protons to modulate the proton beam that is directed at a target area of a patient. Also, the patient specific sparse passive filter can result in the proton beam substantially (defined above) avoiding organs at risk (OARs).

In an aspect, the present disclosure provides for the method of designing patient specific sparse passive filters. The method includes receiving a scan (e.g., computed topography scan, PET scan, MRI scan, x-ray scan, and the like and combinations thereof) of a patient or an area of a patient. An initial geometry of a sparse passive filter based at least in part on the scan can be determined. The initial geometry is determined by applying a ray tracing algorithm to the scan. Additional details are provided in the Examples.

The method also includes determining a dose influence matrix (e.g., how much dose each volume element (voxel) receives from each proton spot) and an LET influence matrix (e.g., the LET_{d} within each voxel for each proton spot), both of which are described in the Examples. In parallel with determining the dose influence matrix and the LET influence matrix, a plurality of geometry variations can be simulated using a particle simulation (as described in the Examples).

The method also includes optimizing output data from the particle simulation to determine an optimized geometry of the patient specific sparse passive filter, the optimization being based at least in part on the dose influence matrix and the LET influence matrix. Optimizing the output data from the particle simulation can also include optimizing a dose of protons from the beam of protons, a dose-averaged dose rate (DADR) of protons from the beam of protons, and dose-averaged linear energy transfer (LET_{d}) of the protons from a beam of protons. Additional details are provided in the Examples.

The method also includes forming the patient specific sparse passive filter. The patient specific sparse passive filter can be produced using 3D printing and made from materials described herein and available from a commercial source.

In an aspect, the present disclosure provides for patient-specific sparse passive filters for simultaneous intensity and energy modulation in proton therapy. The patient-specific sparse passive filter designed by the process of determining an initial geometry of a sparse passive filter based at least in part on a scan of a patient. The process includes applying a ray tracing algorithm to the scan of the patient, and determining the initial geometry based at least in part on a result of the ray tracing algorithm. The process includes determining a dose influence matrix and an LET influence matrix. The process includes simulating a plurality of geometry variations using a particle simulation. The process includes optimizing output data from the particle simulation to determine an optimized geometry; the optimization being based at least in part on the dose influence matrix and the LET influence matrix. The process further comprising receiving the scan of a patient. The process includes fabricating the patient-specific sparse passive filter based, at least in part, on the optimized geometry. Additional details are provided herein and in the Examples.

The process includes optimizing a dose of protons from the beam of protons, a dose-averaged dose rate (DADR) of protons from the beam of protons, and dose-averaged linear energy transfer (LET_{d}) of the protons from a beam of protons from the output data from the particle simulation. The process for determining a dose influence matrix and an LET influence matrix and simulating a plurality of geometry variations using a particle simulation are accomplished in parallel. Additional details are provided herein and in the Examples.

The present disclosure also includes systems for designing a patient-specific sparse passive filter. The system includes at least one computing device comprising a processor and a memory and machine-readable instructions stored in the memory that, when executed by the processor, cause the computing device to perform at least the following functions: receive a scan of a patient; determine an initial geometry of a sparse passive filter based at least in part on the scan; and determine a dose influence matrix and an LET influence matrix. In parallel with determining the dose influence matrix and the LET influence matrix, simulate a plurality of geometry variations using a particle simulation. The system can optimize output data from the particle simulation to determine an optimized geometry, the optimization being based at least in part on the dose influence matrix and the LET influence matrix.

The dose influence matrix and the LET influence matrix can be determined using a Monte Carlo particle an analytical dose engine or an artificial intelligence approach.

The machine-readable instructions which cause the at least one computing device to optimize output data from the particle simulation can further cause the at least one computing device to optimize a dose of protons from the beam of protons, a dose-averaged dose rate (DADR) of protons from the beam of protons, and dose-averaged linear energy transfer (LET_{d}) of the protons from a beam of protons. Also, the machine-readable instructions which cause the at least one computing device to determine an initial geometry, further cause the at least one computing device to apply a ray tracing algorithm to the scan to determine the initial geometry. The machine-readable instructions, when executed, further cause the at least one computing device to at least send the optimized geometry to a fabrication system.

A “processor” or “processing device,” as those terms are used herein encompass an electronic component that is able to execute a computer program or executable computer instructions. References herein to a system comprising “a processor” or “a processing device” should be interpreted as a system having one or more processors or processing cores. The processor may, for instance, be a multi-core processor. A processor may also refer to a collection of processors within a single computer system or distributed amongst multiple computer systems. The term “computer,” as that term is used herein, should be interpreted as possibly referring to a single computer or computing device or to a collection or network of computers or computing devices, each comprising a processor or processors. Instructions of a computer program can be performed by a single computer or processor or by multiple processors that may be within the same computer or that may be distributed across multiple computers.

The term “memory” or “memory device,” as those terms are used herein, are intended to denote a non-transitory computer-readable storage medium that is capable of storing computer instructions, or computer code, for execution by one or more processors. References herein to “memory” or “memory device” should be interpreted as one or more memories or memory devices. The memory may, for example, be multiple memories within the same computer system. The memory may also be multiple memories distributed amongst multiple computer systems or computing devices. More specific examples (a non-exhaustive list) of the computer-readable storage medium would include the following: an electrical connection (electronic) having one or more wires, a portable computer diskette (magnetic), a random access memory (RAM) (electronic), a read-only memory (ROM) (electronic), an erasable programmable read-only memory (EPROM or Flash memory) (electronic), an optical fiber (optical), and a portable compact disc read-only memory (CDROM) (optical). Note that the computer-readable medium could even be paper or another suitable medium upon which the program is printed, as the program can be electronically captured, via for instance, optical scanning of the paper or other medium, then compiled, interpreted or otherwise processed in a suitable manner if necessary, and then stored in a computer memory. In addition, the scope of the certain embodiments of the present invention includes embodying the functionality of the preferred embodiments of the present invention in logic embodied in hardware or software-configured mediums.

### Examples

Now, having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure. In particular, the Examples describe the use of protons, but other energetic entities can be used such as other high energy charged particles (e.g., ions that are heavier than protons (for example, helium, lithium, carbon, or neon atomic nuclei) or electrons). In this regard, the dose, dose rate, and LET for other types of energetic particles can be modulated with a sparse passive filter. Also, the Examples describe FLASH radiotherapy but other non-FLASH therapy can be used with as well.

### Example 1: Forward Heuristic Solution to IPO-IMPT Problem

### 3D Ridge Filter Design

The beam-specific planning target volume (BSPTV) is used to design the patient-specific ridge filters. Each modulation step creates a separate Bragg peak. The weight (cross-sectional area of the step) and thickness (height) of each step are denoted by variables wₖ and tₖ. These are used as inputs to the equation (2.1), where Dᵢ represents the dose at the i-th Bragg peak and B(tₖ, j) is the depth dose of the Bragg peak by the k-th modulation ridge thickness at position j. We generate the B (m, n) matrix of n Bragg curves including m points through Geant4 simulation. The value of m is determined according to the step size of the Bragg curves with a fixed depth. For example, in the patient studies described here, a step size of 0.1 mm and a Bragg peak depth of 40 cm were used, resulting in m=4000. The value of n is determined according to a defined discretized step size of the SOBP length at a specific location. For example, a discretized step size of 0.2 mm and an SOBP of 3 cm, results in a value of n=150. The ridge filter information file is generated by solving the equation set (2.1) using the least square method (equation (2.2)) to provide the area wₖ of thickness tₖ. Importantly, equation (2.2) must be solved for each pin location to obtain the filter pin information.

$$\{ \begin{matrix}{D}_{1} = B ( {t}_{1} , 1 ) {w}_{1} + B ( {t}_{2} , 1 ) {w}_{2} + … B ( {t}_{N} , 1 ) {w}_{N} \\ {D}_{2} = B ( {t}_{1} , 2 ) {w}_{1} + B ( {t}_{2} , 2 ) {w}_{2} + … B ( {t}_{N} , 2 ) {w}_{N} \\ {D}_{M} = B ( {t}_{1} , M ) {w}_{1} + B ( {t}_{2} , M ) {w}_{2} + … B ( {t}_{N} , M ) {w}_{N}\end{matrix} \qquad ( 2.1 )$$

$${arg min}_{w} \frac{1}{2} {‖ B \vec{w} - \vec{D} ‖}^{2} \qquad ( 2.2 )$$

Achieving the desired IMPT-like dose distribution requires that the spatial resolution (the smallest modulation step width) of the filter pins be much less that than Gaussian sigma for the proton spot. This assures that proton energies will be mixed in the desired proportion to enable a smooth SOBP. In some embodiments, this requires a resolution on the order of 100 μm to allow optimization of the smallest ridge weight wₙ.

The single pin optimization is then extended to multiple pins, which are arranged to cover the whole tumor volume. The optimized weight factors are translated to the geometrical parameters of the filter pin. The filter pin positions are defined in the BEV. The complete assembly includes both filter pins and a range compensator. 3D software can then be used to generate a stereolithography file to be sent to a 3D printer for printing. Micro-CT image can be used to demonstrate that the ridge filter conforms to the design. A full Monte Carlo dose calculation engine was developed for the patient-specific ridge filter using Geant4 (Version 10.7) to calculate the patient dose with a ridge filter.

### Treatment Planning System

Once the patient's 3D voxelized geometry file, ridge filter information file, and beam parameters (including gantry angle and initial spot map) are obtained, this information can be fed into a dose calculation engine, such as the Geant4, to obtain the 3D dose and LET influence matrices. These matrices serve as inputs for the inverse optimization of spot weights. The open-source treatment planning toolkit, matRad, was used to develop a treatment planning system (TPS), implementing the IPO-IMPT framework to generate an optimized spot map that conforms to the target dose coverage and OAR constraints specified in the treatment plan. The matRad-based TPS determines the optimized spot map to meet the minimum MU constraint. MatRad is written in MATLAB and relies on an interior point optimization package (IPOPT) to solve the fluence optimization problem. It uses L-BFGS with a logarithmic barrier to implement the required boundary constraints. Specifically, the spot map is optimized based on the equation (2.3), which is the main objective function for optimization. A DADR quadratic term has been added to the original objective function to allow simultaneous optimization of dose, DADR, and LETd. Equation (2.4) describes the dose summation process using the weighted dose influence matrix. Equation (2.5) describes the calculation of DADR. Equation (2.6) describes the calculation of the minimum MU.

$$min f ( w ) = \{ \text{} \begin{matrix}{\sum }_{t ϵ T} \frac{{α}_{t}}{{N}_{t}} {\sum }_{i = 1}^{{N}_{t}} {( max \{ 0 , {η}_{t} − {d}_{i} ( w ) \} )}^{2} + {\sum }_{o ϵ OAR} \frac{{α}_{o}}{{N}_{o}} {\sum }_{i = 1}^{{N}_{o}} {( max \{ 0 , {d}_{i} ( w ) − {η}_{o} \} )}^{2} + \\ {\sum }_{o ϵ OAR} \frac{{α}_{o , D A D R}}{{N}_{o}} {\sum }_{i = 1}^{{N}_{o}} {( max \{ 0 , D A D {R}_{o} − D A D {R}_{i} ( w ) \} )}^{2} + \\ {\sum }_{o ϵ OAR} \frac{{α}_{o , L E T}}{{N}_{o}} {\sum }_{i = 1}^{{N}_{o}} {( max \{ 0 , L E T {d}_{i} ( w ) − L E T {d}_{o} \} )}^{2}\end{matrix} \qquad ( 2.3 )$$

$${d}_{i} = {\sum }_{j} {D}_{i , j} {w}_{j} \qquad ( 2.4 )$$

$$DAD {R}_{i} = \frac{1}{{D}_{i}} {\sum }_{j = 1}^{{N}_{o}} ( {D}_{i j} {w}_{j} ) ( {D}_{i j} {I}_{j} ) \qquad ( 2.5 )$$

$${w}_{j , min} = \frac{{I}_{n o z z l e} \times {T}_{min}}{{N}_{M U}} \qquad ( 2.6 )$$

$$LE {T}_{{d}_{i}} = \frac{1}{{D}_{i}} {\sum }_{j = 1}^{{N}_{o}} {L}_{i j} {D}_{i j} {w}_{j} \qquad ( 2.7 )$$

Here, nₜ & nₒ are the reference doses, Nₜ & Nₒ are the number of voxels, and αₜ & αₒ are the penalty factors for target and OAR, respectively. Values for dᵢ and DADRᵢ are given by equations (2.4) and (2.5), respectively. Dᵢⱼ is the influence matrix of dose, Lᵢⱼ is the influence matrix of LET_{d}, w is the spot weight in MU, i is the voxel index, and j is the spot index. I_{nozzle}, Tₘᵢₙ & N_{MU} are nozzle current, minimum spot duration and number of protons per MU, respectively.

### Sparse Ridge Filters (Also Referred to as “Sparse Passive Filter”)

Regular ridge filters, designed using the IPO-IMPT framework provide increased DADR for some OARs while maintaining tumor coverage. However, the optimization does not take depth modulation into account. Sparse ridge filters, from which some pins are omitted, provide a means to further increase the DADR for optimal FLASH sparing. Removing filter pins at specific locations preserves a higher proton flux, while the remaining filter pins still provide adequate SOBP dose coverage to the BSPTV.

To generate the sparse ridge filters, the dose influence matrices are calculated for a regular ridge filter and for a range compensator alone with no pins. The filter pin location map is used as the proton spot map, so that the dose of each beamlet reflects the contribution of a specific ridge filter pin. Using these two dose influence matrices, it is possible to obtain an optimized IPO-IMPT plan. The spot weighting factors can then be derived as well. If the pin at location j results in

$$\frac{{w}_{j}^{r}}{{w}_{j}^{r} + {w}_{j}^{t}} > {f}_{j} ,$$

where fⱼ is a user-defined threshold, wⱼ^{T} is the weighting factor for filter pin location j of the regular ridge filter and wⱼ^{t} is the weighting factor for pin location j of the filter compensator, the pin is kept; otherwise, the pin is removed. The sparse ridge filter is generated from this process. After the pin locations are selected, the sparse ridge filter design is generated. The sparse filter design allows higher DADRs for OARs, including lung and heart.

### Example Filters Design and Treatment Plans

To demonstrate the IPO-IMPT framework, we designed ridge filters and developed treatment plans for three example lung cancer patients. Patient-specific ridge filter and range shifter assemblies were designed to achieve conformal target dose coverage using a 250 MeV proton beam. The BSPTV was created with 5% range uncertainty and 5 mm setup uncertainty. For our scanning beam proton therapy system, using a minimum duration of 1 millisecond and a constant current 300 nA, a value of 300 was taken as the minimum MU. The clinical target volume (CTV) received a prescribed dose of 50 Gy (10 Gy×5 fractions) with a maximum allowable dose for hotspots corresponding to 125% of the prescription dose (62.5 Gy). For all three patients, lung and heart were considered as OARs. For Patients 2 and 3, esophagus was also considered. Three beam angles were considered for each patient.

IPO-IMPT plans were generated for regular and sparse ridge filters at each beam angle and compared with conventional IMPT plans, as detailed in the Results section. A preliminary dose verification with a patient-specific ridge filter was also conducted through the experiment as detailed in the Result section. To generate the evaluating structures, Heart_eva and Lung_eva, we first created a uniform 5 mm expansion of the BSPTV. The 5 mm BSPTV expansion was chosen to include the gradual dose fall off beyond the BSPTV, recognizing that the dose within this margin region may exceed the lower threshold for a FLASH effect. Next, the CTV was removed from the expanded BSPTV and Lung_eva was defined as the overlap between this and the lung. The Heart_eva and Esophagus_eva structures were generated using a similar approach. The rationale for using only the defined Heart_eva, Lung_eva, and Esophagus_eva volumes, rather than the whole heart and lung, was that evaluation of a very large structure might mask the significance of a high dose or high dose rate due to a large volume with a low dose and low dose rate. For multiple beam plans, the overall evaluating structure is the Boolean union of the evaluation structures for each beam.

For each plan, the distribution of dose, DADR, and LETd were calculated and corresponding volume histograms were generated. The FLASH effect has been reported to have a dose threshold between 4 Gy to 10 Gy 39-42. Here, 4 Gy per fraction per field was used as a conservative estimate. The FLASH dose rate threshold has been reported to be between 40 and 100 Gy/s. Here, 40 Gy/s was used. Each field independently meets the dose and dose rate constraints for the FLASH effect. These thresholds can be modified as knowledge of the FLASH effect improves.

For generating the dose rate volume histograms, the DADR were assigned as zero for the voxels that do not meet the dose threshold. Thus, the fraction of volume achieving FLASH can be directly observed by inspection of the DADR volume histogram, as only the voxels that meet the dose and dose rate thresholds contribute to the histogram.

IPO-IMPT with Regular Ridge Filters

To demonstrate the functionality of the IPO-IMPT framework, regular ridge filters were designed and treatment plans for three sample lung cancer patients were developed. Patient 1 had a central lung tumor, very close to the heart. Heart and uninvolved lung were OARs. Patient 2 had a metastatic tumor in the right lower lobe and Patient 3 had a tumor in the subcarinal lymph node. The esophagus was an additional OAR in both these patients.

A single-beam IPO-IMPT plan was generated for Patient 1, with a primary goal of reducing LETd to heart while maintaining target coverage. The target coverages for the IPO-IMPT and IMPT plans are similar. However, the IPO-IMPT framework resulted in a marked reduction of LETd in the heart. For comparison, a multi-beam plan was constructed for the same patient, where the primary goal was to optimize DADR, while maintaining adequate dose and LETd optimization. Together, the results demonstrate that adoption of the IPO-IMPT framework, in combination with regular ridge filters, results in at least modest improvements to DADR and LETd for OARs, while maintaining tumor coverage and meeting other constraints.

Patients 2 and 3 were chosen to illustrate the potential to spare the esophagus. Using the regular ridge filter approach, almost 100% of the Esophagus_eva structure meets the 40 Gy/s FLASH threshold. This very high coverage was seen with both IPO-IMPT or IMPT. For Patient 2, IPO-IMPT modestly decreased LETd for heart and esophagus and increased in DADR for heart (97% FLASH coverage with IBO-IMPT versus 90% for IMPT).

Dose distributions of FLASH plans and conventional IMPT plans were also compared for each patient. Dose distributions to the CTV and OARs are generally comparable. For completeness, the FLASH plans were also compared to the original VMAT plans for Patients 2 and 3. Again, dose distributions are comparable.

IPO-IMPT with Sparse Ridge Filters

Regular ridge filters, originally designed for dose optimization only, cannot fully realize the benefits of the IPO-IMPT framework, because spot-specific dose-depth modulation is not optimized. To address this, sparse ridge filter designs were explored, in which some pins are removed using the heuristic decision process described above.

For Patient 1, a fully optimized IPO-IMPT plan was generated with sparse ridge filters and multiple beams. An IMPT-optimized plan using regular ridge filters was used for comparison. The OAR optimization constraints for the two plans were the same. Tumor coverage was maintained and hotspots were well controlled with both plans. However, the IPO-IMPT plan based on sparse ridge filters results in a marked improvement to DADR in the OARs. The volume that received a dose rate of ≥40 Gy/s increased by 31% for Heart_eva and by 50% for Lung_eva 50%. The LET_{d} for the two plans was substantially the same. Together, results show that the use of sparse ridge filters and multiple beams helps realize the full potential of the IPO-IMPT framework.

A separate set of optimized single-beam plans using sparse ridge filters was demonstrated. The increased DADR for lungs using sparse ridge filters versus regular ridge filters is evident. The individual plans have some hotspots within BSPTV (which slightly exceed the 125% prescription dose), but sequential delivery as SBRT fractions reduces these and improves target coverage. Together, dose coverage is similar to the multi-field plan, but with better FLASH sparing due to increased volumes that meet the 40 Gy/s dose threshold in each field and fraction.

Sparse ridge filter-based plans were also developed for Patients 2 and 3. A comparison of IPO-IMPT optimized plans based on regular and sparse ridge filters was conducted. The FLASH plan with a sparse ridge filter further increases the DADR to the esophagus while maintaining similar tumor coverage and meeting other constraints.

Preliminary Dose Verification with a Patient-Specific Ridge Filter

To verify the ability of the ridge filter assembly to deliver the predicted dose, proton dose measurements were performed. The ridge filter assembly, which includes filter pins and a compensator, was placed on the TO beam axis. A range shifter, solid water, and an ionization chamber array were placed downstream. A treatment plan optimized for Patient 1 and designed to provide a uniform dose to the CTV was delivered. The calculated dose distribution was 25 mm depth from the solid water surface. The total gamma passing rate was 92.9% (3 mm/3%, 10% threshold) for the absolute doses, which exceeds the standard patient QA passing criteria of 90%. Results provide a preliminary demonstration that the ridge filter assembly can facilitate the delivery of a clinically acceptable dose distribution. Measurement of dose rate and LET, using a novel time-resolved and spatially resolved detector, is in progress.

### Discussion

In this feasibility study, the use of the IPO-IMPT framework and optimized ridge filters were shown capable of improving DADR and LETd, for lung and heart, relative to a plan generated using a standard IMPT approach. The IPO-IMPT framework, which explicitly incorporates objective functions of dose, DADR, and LETd, provides degenerate solutions for patient-specific ridge filter and spot maps while providing an ability to study the contribution of each term.

Optimization of DADR and LETd, while maintaining a similar dose distribution, is crucial for disentangling the biological contributions of DADR and LET from that of dose per se. For OARs such as great vessels, which have a maximum tolerated dose close to the prescription dose, increasing the DADR above the FLASH threshold (≥40 Gy/sec) may be the best selection. Alternatively, lower dose or LET might be a better option for OARs, such as spinal cord, which have a maximum tolerated dose that is smaller than the prescription dose. Such options can be explored using the IPO-IMPT optimization framework.

Ridge filters have been used previously in particle therapy to avoid the need to switch energy layers, reducing treatment time. They have gained new popularity in the era of FLASH therapy. The work described herein, embodies several further advances, including a flexible scheme for simultaneous optimization of competing objectives of dose, DADR, and LETd, providing multiple solutions. The work also introduces sparsity, that is, ridge filters from which some pins are omitted, to further optimize dose rate and thus FLASH coverage. Preliminary experimental validation is also presented.

The sparse ridge filters are more efficient than regular filters, providing more flexibility to improve the DADR. Use of the sparse filters can lead to some hotspots within the CTV, although this can be mitigated by alternating the beam orientation over SBRT fractions. Different user-defined thresholds for pin removal can lead to different filter designs. A threshold of 50%, provides reasonably good results for large tumors (such as Patient 1), whereas a threshold of 30% was a good starting point for smaller targets (such as Patient 2 and 3). The sparse ridge filter design process is currently based on a heuristic method, where several trial-and-error iterations are generally required to achieve an acceptable result. In some embodiments, a faster dose calculation engine may be used for patient-specific ridge filters, which would allow a combination of the ridge filter and plan optimization processes through a stepwise optimization scheme or using mixed-integer programming. This would allow simultaneous optimization of the proton spot map and the filter pin location map.

It is important to note that the biological mechanism of FLASH sparing remains a subject of active investigation. IPO-IMPT optimization can assist this work by enabling biologists to separate the contribution of LET from dose rate effects. With the IPO-IMPT framework, different beam designs can be examined in parallel to determine the contribution of each term. When better biological models of the FLASH effect are available, the IPO-IMPT can be extended to incorporate them directly, rather than indirectly via DADR and LETd terms. Other examples include replacing the DADR with other dose rate approaches in IPO-IMPT. In some embodiments, a constant beam current is assumed, which allows a simplified optimization model for DADR, keeping spot MUs as the sole decision variables. In some embodiments, solutions for adding current as a decision variable are integrated into the IPO-IMPT framework.

### Example 2: SIEMAC Approach to Solving the IPO-IMPT Problem

Extending the Traditional IMPT Optimization Problem to Solve IPO-IMPT Previously, ziggurat-shaped pins have been used to create SOBPs. However, the present disclosure provides a simpler square pyramid-shaped pin to create the SOBPs to reduce computational effort in design. The objective function is expanded to include dose rate and LET objectives. Thus, the new problem becomes:

$$\begin{matrix}arg min \\ w , {ℓ}_{b} , {ℓ}_{p}\end{matrix} f ( w , {ℓ}_{b} , {ℓ}_{p} ) \qquad ( 1 )$$

$$where$$

$$f ( w , {ℓ}_{b} , {ℓ}_{p} ) = {\sum }_{n} {P}_{n} {f}_{n}^{D} ( w , {ℓ}_{b} , {ℓ}_{p} ) + {\sum }_{n} {p}_{n} {f}_{n}^{D R} ( w , {ℓ}_{b} , {ℓ}_{p} ) + {\sum }_{n} {p}_{n} {f}_{n}^{L E T} ( w , {ℓ}_{b} , {ℓ}_{p} ) . \qquad ( 2 )$$

The dose rate and LET objectives, fₙ^{DR} and fₙ^{LET}, can be easily defined in a way directly analogous to Equation 16, and the arguments are again typically constrained by upper and lower bounds. More specifically, the objective function used in this analysis is

$$f ( w , {ℓ}_{b} , {ℓ}_{p} ) = {\sum }_{B S P T V} \frac{{p}_{B S P T V}}{{N}_{B S P T V}} {\sum }_{i ∈ BSPTV} Θ ( \frac{\hat{d}}{{N}_{f}} - {d}_{i} ) {( {d}_{i} - \frac{\hat{d}}{{N}_{f}} )}^{2} + \frac{{p}_{CTV}}{{N}_{C T V}} {\sum }_{i ∈ CTV} Θ ( \hat{d} - {d}_{i} ) {( {d}_{i} - \hat{d} )}^{2} + {\sum }_{O A R} \frac{{p}_{O A R}}{{N}_{O A R}} {\sum }_{{i ∈ OAR ,}_{{d}_{i} > {D}_{0}}} Θ ( - {DR}_{t} ) {( {DR}_{i} - )}^{2} + {\sum }_{O A R} \frac{{p}_{O A R}}{{N}_{O A R}} {\sum }_{{i ∈ OAR ,}_{{d}_{i} > {D}_{0}}} Θ ( {LET}_{i} - ) {( LE {T}_{i} - )}^{2} + \frac{{p}_{R O B}}{{N}_{R O B}} {\sum }_{i ∈ ROB} {d}_{i}^{2} \qquad ( 3 )$$

subject to upper and lower bounds on each optimization variable:

$$v \text{?} < v \text{?} < v \text{?} \qquad ( 4 )$$

$$\text{?} \text{indicates text missing or illegible when filed}$$

where the generic variable ν has been introduced for simplicity to represent the concatenation of w, l_{b}, and lₚ; and d, DR, and LET are the prescription dose, target dose rate, and target LET, respectively; and Θ is the Heaviside function. D₀ is a dose cutoff, where voxels with a dose below this value are not considered in the objective; typical values are 5%-10% of the prescribed dose. ROB refers to the rest-of-body which is everything in the body besides the CTV and BSPTVs. Since dose rate and LET have contributions from each spot, a dose averaged dose rate and LET are used, i.e.,

$$D {R}_{i} = \frac{{\sum }_{j} {D}_{ij}^{2} {w}_{j} {I}_{j}}{q \text{?} {\sum }_{j} {D}_{i j} {w}_{i}} \qquad ( 5 )$$

$$and$$

$${LET}_{i} \approx \frac{{\sum }_{j} {LET}_{i j} {D}_{i j} {w}_{j}}{{\sum }_{j} {D}_{i j} {w}_{j}} \qquad ( 6 )$$

$$\text{?} \text{indicates text missing or illegible when filed}$$

where DRᵢ is the DADR in voxel i, Iⱼ is the nozzle current of spot j (i.e., 300 nA in some embodiments), LET; is the dose averaged LET in voxel i, and LETᵢⱼ (the LET influence matrix) is the dose averaged LET in voxel i due to spot j.

A restricted influence grid (RIG) is introduced to limit the extent of dose and LET influence matrices by inclusion of the spots that are within FLASH millisecond timing proximity of the location of the highest instantaneous dose. A RIG exists for each voxel i, and includes voxel i plus the neighboring voxels surrounding it. A time value for each RIG can then be defined as

$$t \text{?} = \frac{\text{?} {\sum }_{j} {w}_{j} {f}_{ij}}{I} = {\sum }_{j} {f}_{i j} {t}_{j}^{spot} \qquad ( 7 )$$

$$\text{?} \text{indicates text missing or illegible when filed}$$

where fᵢⱼ is the fraction of spot j that impinges on RIG i and

$${t}_{j}^{spot} = {q}_{e} {w}_{j} / I$$

is the actual time duration of spot j. Alternatively, fᵢⱼ could also be defined as a Boolean value equal to 1 when the threshold of 0.5 is met, and 0 otherwise. In other words, fᵢⱼfⱼ^{spot} is the hypothetical irradiation time on RIG i from spot j assuming the entire spot impinges on RIG i rather than just a fraction of it, and tᵢ^{RIG} is a sum over these values without accounting for scan time+delivery time of other spots. Thus,

$${t}_{i j}^{RIG} \approx {t}_{j}^{spot}$$

when the spot and RIG mostly overlap, and

$${t}_{i j}^{RIG} << {t}_{j}^{spot}$$

when the spot and RIG overlap very little.

For simplicity, a very rudimentary version of a RIG is used as well as a restricted dose (or LET) influence matrix:

$${D}_{i j} = \{ \begin{matrix}{D}_{i j}^{unrestricted} & i ∈ spot specific restricted influence grid \\ 0 & otherwise\end{matrix} \qquad ( 8 )$$

where

$${D}_{i j}^{unrestricted}$$

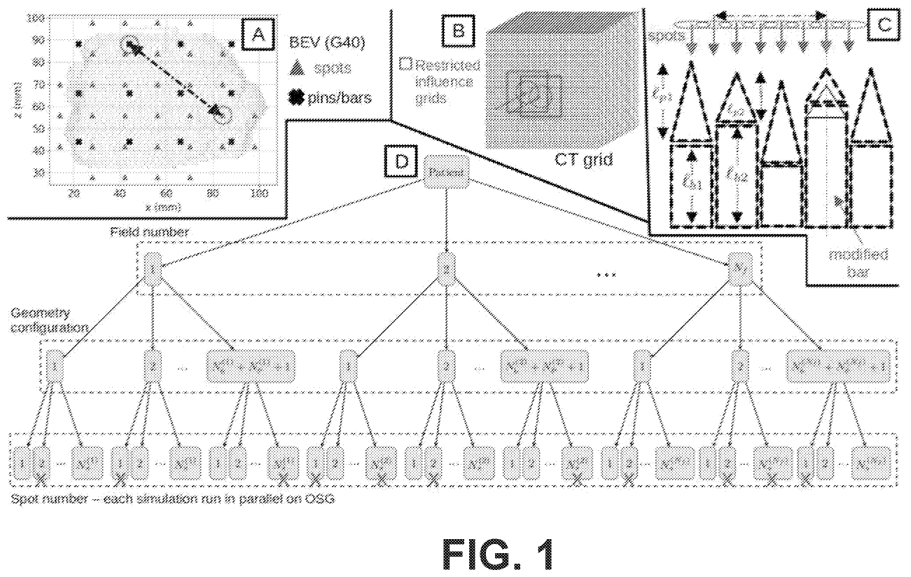

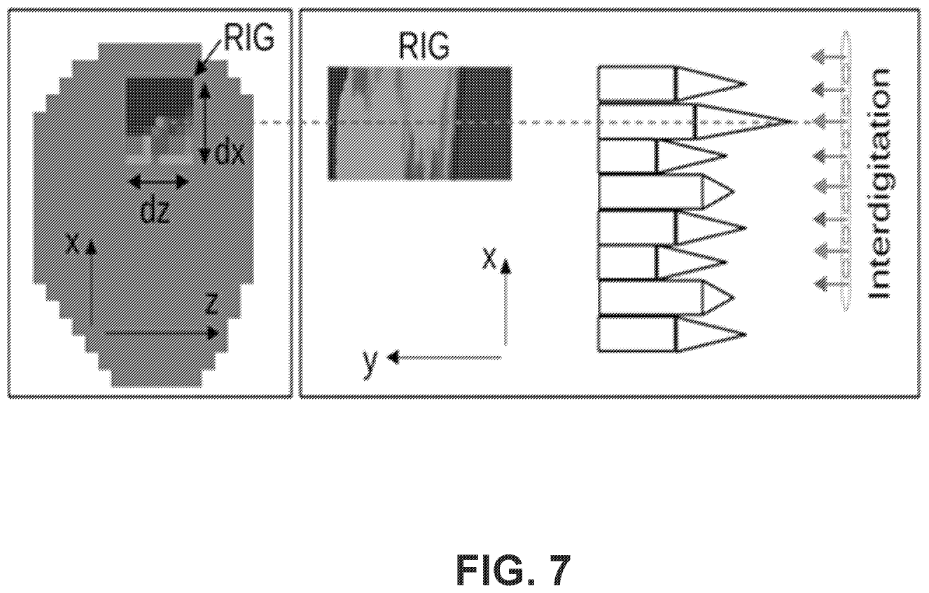

is the dose to voxel i due to spot j considering the entirety of the CT grid. The restricted Dᵢⱼ, illustrated in FIGS. 1B and 7, significantly trims down the CT grid for the sake of computational performance by assuming the dose is negligible in voxels far away from the spot. The dot-dash line with double-ended arrows in FIGS. 1A and 1C show the spots interjoining with sparse pins/bars subject to RIG.

These changes to the optimization problem present several challenges. First, the added arguments and objectives make the problem more complex and make solving the problem more CPU intensive. Furthermore, Lᵢⱼ must also be calculated in addition to Dᵢⱼ. Second, the added arguments and objectives can make the problem non-convex. Third, the variability of the geometry parameters means that Dᵢⱼ and Lᵢⱼ need to be re-calculated many times as the geometry changes, and it also makes the gradient calculation much more difficult since Dᵢⱼ and Lᵢⱼ are not constant. This leads to a further and very significant increase in necessary computing power.

### Implementation of the SIEMAC Approach

To address these challenges, a parallel computing framework is used. The first step is to define the initial (i.e., zeroth order) geometry. In some embodiments, this can be done using ray tracing to design a filter meant to produce a conformal dose distribution. In some embodiments, the initial geometry is defined using a forward heuristic, such as the sparse modulation technique. In other embodiments, the initial geometry is defined using a global search algorithm such as differential evolution, dual annealing, or other global search algorithm as can be appreciated.

Next, a quasi-Newton method (the Limited-memory Broyden-Fletcher-Goldfarb-Shanno B (L-BFGS-B) algorithm) is used to better optimize the initial geometry, along with the spot weights. The gradient of the objective function is

$$∇ f = ( \frac{\partial f}{\partial {w}_{1}} , ⋯ , \frac{\partial f}{\partial {w}_{N} \text{?}} , \frac{\partial f}{\partial ℓ \text{?}} , ⋯ , \frac{\partial f}{\partial ℓ \text{?}} , \frac{\partial f}{\partial {ℓ}_{p 1}} , ⋯ , \frac{\partial f}{\partial {ℓ}_{p \text{?}}} ) . \qquad ( 9 )$$

$$\text{?} \text{indicates text missing or illegible when filed}$$

### The Partial Derivatives

$$\frac{\partial f}{\partial {w}_{1}} \frac{\partial f}{\partial {w}_{N} s}$$

are straightforward to calculate analytically. The remaining partial derivatives are estimated using the finite difference approximation

$$\frac{\partial f}{\partial {v}_{1}} = \frac{f ( v \text{?} , … , v \text{?} + Δ v \text{?} , … , v \text{?} ) − f ( v )}{Δ v} . \qquad ( 10 )$$

$$\text{?} \text{indicates text missing or illegible when filed}$$

Since f depends on Dᵢⱼ and Lᵢⱼ, and since Dᵢⱼ and Lᵢⱼ depend on the pin and bar lengths, it can be seen in Equations 9 and 10 that the number of geometries, and therefore the number of Dᵢⱼ's and Lᵢⱼ's that need to be calculated with MC, is N_{b}+Nₚ+1 for each field and for each iteration of the optimization.

In order to complete these calculations in a reasonable amount of time, they were broken down into parallelizable pieces and submitted to a computing cluster or supercomputer. The exact parallelization scheme is illustrated in FIG. 1D. A red X in the bottom row of FIG. 1D represents a simulation that can be skipped due to the spot being far away from the modified geometry component (represented by the dot-dash line with double-ended arrows in FIGS. 1A and C), which therefore saves time.

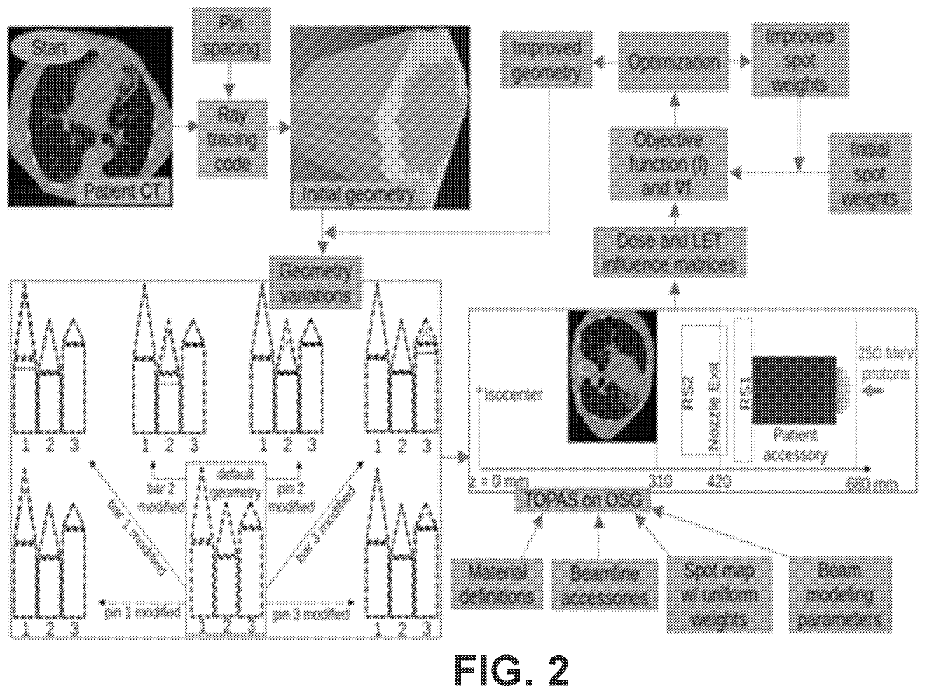

The overall workflow of the optimization can be seen in FIG. 2. The process begins by using a ray tracing algorithm with a patient's computed tomography (CT) scan to define the initial geometry of the pins and bars. A Monte Carlo tool (such as TOPAS MC), analytical engine, artificial intelligence, or other approach is then used to calculate Dᵢⱼ and Lᵢⱼ. In parallel, many geometry variations are also simulated that are needed to calculate the gradient of the objective function. The simulation output data are then fed into an optimization algorithm and the process is repeated until an acceptable solution is reached.

### SIEMAC for Preclinical Applications

Unlike clinical treatment plans, preclinical studies typically aim to deliberately irradiate an OAR and therefore require different optimization objectives. Preclinical objectives should include minimizing the spreads of the dose, dose rate, and LET distributions in the OAR target, thereby minimizing uncertainty when separating the contributions from each of these quantities on extra biological dose (XBD). The SIEMAC algorithm was tested to see if it is feasible to indirectly optimize XBD via the physical quantities of dose, dose rate, and LET. In an example, the objective function, Equation 11, was first set to deliver a uniform dose of 20 Gy to the target, which represents the threshold for short-term pneumonitis and long-term fibrosis. In other words, the last two lines of Equation 11 were not used initially.

$$f ( w , {ℓ}_{b} , {ℓ}_{p} ) = \frac{{p}_{TARG}}{{N}_{TARG}} {\sum }_{i ∈ TARG} {( {d}_{i} - \hat{d} )}^{2} + \frac{{p}_{R O B}}{{N}_{ROB}} {\sum }_{i ∈ ROB} {d}_{i}^{2} + \frac{{p}_{TARG}}{{N}_{TABG}} {\sum }_{i ∈ TARG} {( D {R}_{i} - )}^{2} + \frac{{p}_{TARG}}{{N}_{TABG}} {\sum }_{i ∈ TARG} {( {LET}_{i} - )}^{2} \qquad ( 11 )$$

Then, a second round of optimization was done that included the last two lines of Equation 11 to attempt to narrow the dose, DADR, and LETd distributions, and therefore reduce the uncertainty in these quantities, while maintaining similar target dose coverage. The magnitude of spreads of dose, dose rate and LET distributions and their XBDᵢ(DADR) and XBDᵢ(LET) on a 36-mm spherical irradiation target of a minipig lung were compared before and after IPO-IMPT. XBD can be XBD; (DADR) and XBD; (LET) (where i is the voxel number), defined as

$$X B {D}_{i} ( D ADR ) = {d}_{i} \frac{k}{1 + {e}^{− a} \text{?}} \qquad ( 12 )$$

$$and$$

$$X B {D}_{i} ( LET ) = {d}_{i} \text{?} {LET}_{i} \qquad ( 13 )$$

$$\text{?} \text{indicates text missing or illegible when filed}$$

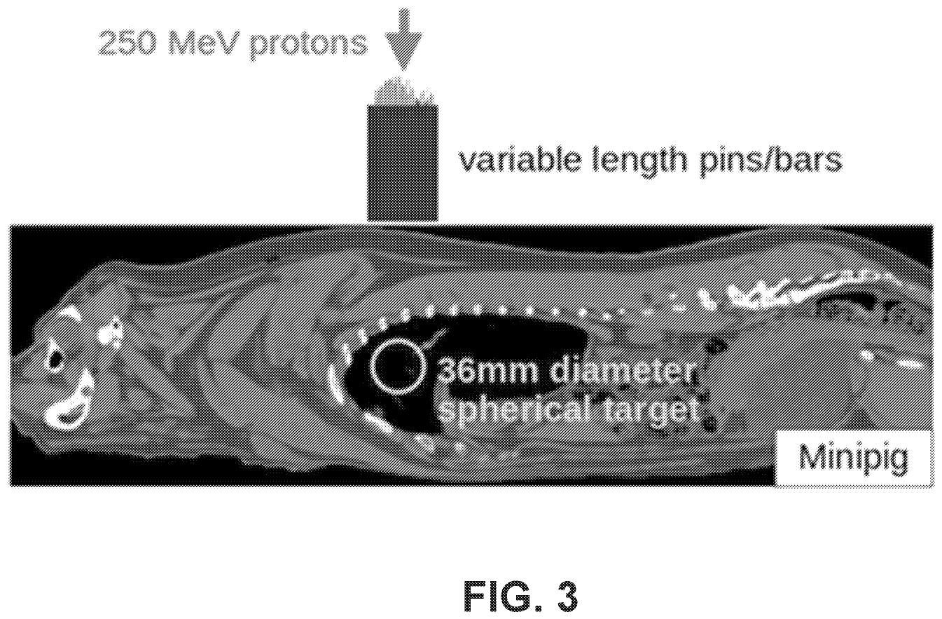

which represent adjustments to the physical dose that take into account biological responses to radiation. Advantages to healthy tissue are represented by larger values of XBD (DADR) and smaller values of XBD (LET). Here, a, k, DRₜ, and c are parameters that depend on biological mechanisms. Optimizing the pin and bar lengths can improve sparse compensation and sparse modulation, along with improved spot maps. The design of the minipig simulations is shown in FIG. 3, which includes an anterior 250 MeV proton pencil beam and sets of variable length pins and bars that can be optimized to irradiate the spherical target.

Traditional IMPT optimizes the weights (w) of a pencil beam spot map in order to produce a conformal dose distribution. A brief summary of traditional IMPT optimization is described below where important quantities such as the dose influence matrix (Dᵢⱼ), objective function (f), prescribed dose (d), and penalty factor (p) are also defined.

Traditional IMPT optimization consists of solving the problem

$$arg min \text{?} f ( w ) \qquad ( 14 )$$

$$\text{?} \text{indicates text missing or illegible when filed}$$

where w are the spot weights, N_{S} is the number of spots, and

$$f ( w ) = {\sum }_{n} {p}_{n} {f}_{n}^{D} ( w ) \qquad ( 15 )$$

where f is the overall objective function, and fₙ^{D} are the individual dose objectives with relative weights (or “penalties”) pₙ. The solution to the optimization problem is usually bounded by upper and lower limits on the spot weights (e.g., positivity or minimum MU constraints).

Many different dose objectives can be defined. For example, one common one is the squared deviation objective

$$f \text{?} = \frac{1}{{N}_{v}} {\sum }_{i ∈ S} {( {d}_{i} - \hat{d} )}^{2} \qquad ( 16 )$$

$$\text{?} \text{indicates text missing or illegible when filed}$$

where S is the set of voxels within a given structure (e.g., tumor, heart, lungs, etc.), N_{V} is the number of voxels in S, dᵢ is the dose to voxel i, and d is the prescribed dose. This objective penalizes the overall objective function every time a voxel's dose deviates from the prescription, with larger deviations leading to larger penalties.

The dose to a given voxel, dᵢ, requires the dose influence matrix, Dᵢⱼ, which gives the dose per particle to voxel i due to spot j, to be known, i.e.

$${d}_{i} = {\sum }_{j} {ω}_{j} {D}_{ij} \qquad ( 17 )$$

where wⱼ is the weight of, or number of particles in, spot j. Dᵢⱼ is typically calculated using a MC simulation or an analytical dose engine, with MC being preferable. While this calculation can be CPU intensive, it is not in general problematic given modern computing power, and it only needs to be performed once, since Dᵢⱼ is a constant in this context. This sort of optimization problem usually represents a convex problem and, once Dᵢⱼ is known, it can be solved fairly easily using standard optimization techniques.

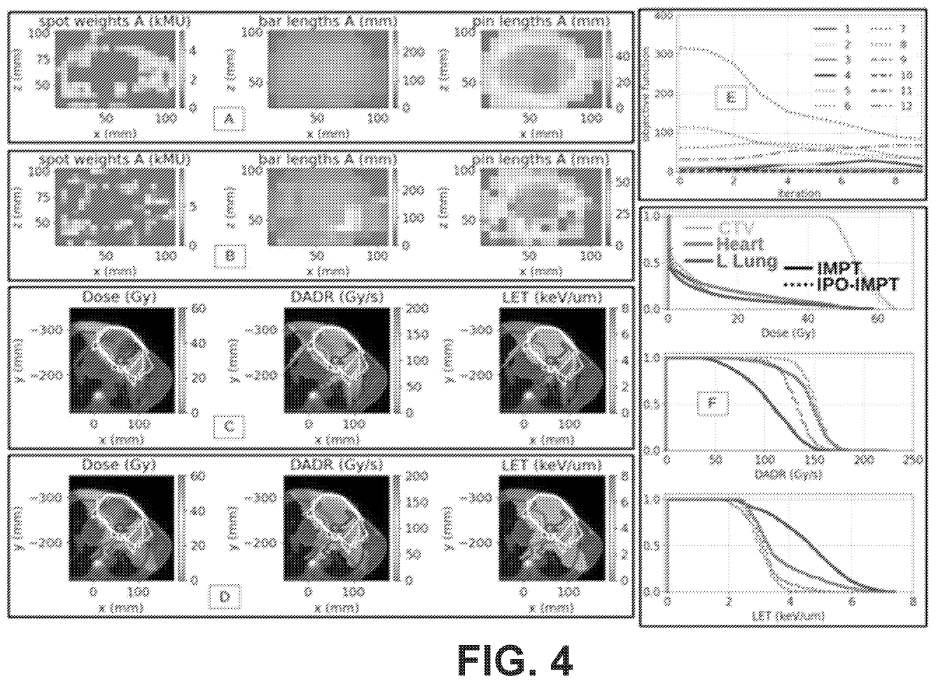

The arguments of the objective function are expanded to include geometry parameters. Specifically, l_{b}∈R^{N}^{_{b}} are the lengths of the range compensating bars (bars for short) with N_{b} representing the number of bars, and lₚ∈R^{N}^{ₚ} are the lengths of the range modulating pins (pins for short) with Nₚ representing the number of pins, as shown in FIG. 1A. Usually, N_{b}=Nₚ, but this is not strictly necessary, so they are kept as two separate variables. A summary of these geometry components can be found in FIG. 4. FIG. 1A also shows the interjoining of spots and pins, i.e., the red spots impinge on pin peaks and the blue spots impinge on the valleys. Furthermore, since IMPT is typically delivered using multiple fields, superscripts are used on the variables to identify to which field that variable belongs (e.g.,

$${N}_{b}^{( 2 )}$$

is the number of bars for field number 2) and use N_{f} to represent the total number of fields.

### SIEMAC Lung Cancer Patient Plan

To demonstrate SIEMAC, a three-field treatment plan was created for a representative lung cancer patient. The dose prescription to the centrally located CTV was 50 Gy, with nearby OARs including the heart and left lung. FIG. Error! Reference source not found. summarizes the result. Panel A shows the spot map, bar lengths (e.g., 1 mm to 100 mm), and pin lengths (e.g., 1 mm to 100 mm) for one of the three fields used (field A=gantry 40, field B=gantry 0, field C=gantry 320) in this study for iteration 0. Iteration 0 is defined to be the result after spot-weight-only optimization (i.e., the geometry parameters are held fixed) has been done using traditional IMPT techniques. Panel B is the same as panel A except after 9 iterations. Similarly, panels C and D show before and after distributions of dose, dose rate, and LET for an axial slice of the patient. Panel E shows the different components of the objective function vs the optimization iteration number. Finally, panel F shows dose, dose rate, and LET volume histograms for the CTV, left lung, and heart.

The plots in FIG. 4F show sizeable improvements to the dose rate and LET distributions in the lung and heart, with a negligible sacrifice to the dose distributions, when comparing traditional IMPT to IPO-IMPT with SIEMAC. For the OARs, we use an evaluation volume, which refers to the overlap between the OAR and BSPTV, excluding the CTV and any voxels with dose below 4 Gy. For the heart and lung, the percentage of the evaluation volume receiving above the FLASH threshold of 100 Gy/s rose from 93% to 100% and from 57% to 96%, respectively. Additionally, LET_{d} coverage above 4 keV/μm dropped from 68% to 9% in the lung and from 26% to <1% in the heart. These improvements can be attributed to the shortening of up to 100 mm bar length and 60 mm pin length (FIG. 4A-B), demonstrating improvements to sparse compensation and sparse modulation, along with improved spot maps that were optimized simultaneously with the pin and bar lengths. The sparse compensation in particular might explain the improvements in the LET distribution over our initial forward heuristic solution.

### Preclinical Optimization

To demonstrate this functionality of SIEMAC, a single field plan has been generated for an animal irradiation with narrower dose, dose rate, LET, and XBD distributions in the irradiated OAR compared to an unoptimized plan, therefore reducing the uncertainty in these variables when deriving XBD models from preclinical studies. Values of c=0.04 μm/keV, DRₜ=40 Gyls, k=0.5, and a=4/DRₜ, were used in equations 12 and 13.

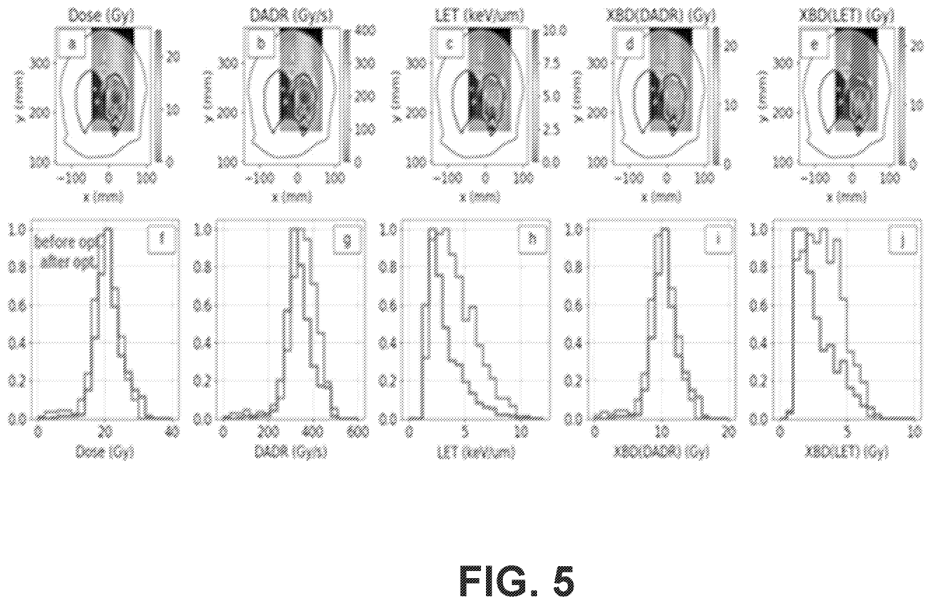

FIG. 5 summarizes the results of the SIEMAC-generated plan, which shows dose, dose rate, LET, XBD (DADR), and XBD (LET) distributions for the target in the left lung of the minipig before and after optimization. The improvements to the optimized plan are quantified by reporting both the full-width-half-maximum (FWHM) of each distribution, which is large for undesirable widely spread distributions and approaches zero for ideal distributions, as well as the area under the histograms after normalizing to a maximum value of 1, which, similarly, is large for undesirable distributions and smaller for ideal distributions. The results show that SIEMAC can be used to reduce the unoptimized wide spread in dose, DADR, and LET_{d} (red vs blue lines in FIG. 5 panels f-j) distributions in animal studies.

For dose, SIEMAC decreased the FWHM by 30% (10 Gy to 7 Gy) and the area of the normalized histogram by 15% (4.8 to 4.1 a.u.). For DADR, the FWHM decreased by 1.2% (122 Gy/s to 120 Gy/s) and area decreased by 21% (4.8 to 3.8 a.u.). And for LET, FWHM decreased by 57% (7.1 keV/μm to 4.0 keV/μm) and area decreased by 44% (7.1 to 4.0 a.u.). To associate extra toxicity (i.e. biological effect) due to dose rate and LET distributions, XBD (DADR) and XBD (LET) are calculated using the proposed XBD model described in equations 12 and 13. The inverse solution of IPO-IMPT demonstrated a modest reduction of XBD (DADR) because the optimization algorithm considers the unoptimized DADR is well above the full UHDR benefit of 100 Gy/s (FIG. Error! Reference source not found.g), at 300 nA nozzle current. Such inverse solution of IBO-IMPT can improve much more XBD (DADR) for other organs and other beam conditions when needed as demonstrated for XBD (LET) (FIG. Error! Reference source not found.h). In summary, the results show a sizable XBD (LET) with a wide FWHM and area without optimization therefore XBD (LET) must be considered and optimized when studying UHDR sparing of lung toxicity.

These results demonstrate a proof-of-concept that SIEMAC can be used to produce proton FLASH treatment plans that provide considerable improvements over existing planning algorithms (FIG. 4 for clinical results and FIG. 5 for preclinical results). The inverse SIEMAC solution improves upon the initial forward heuristic solution by iteratively optimizing range modulation, range compensation, and spot intensity map. The solution provides an opportunity to modulate sub-spot proton energy and proton intensity, which are vital for microscale radiation transport and thus FLASH optimization for simultaneous improvements in dose rate and LET of OARs. The technique has been shown to also be useful in animal studies for narrowing the dose, dose rate, and LET distributions in the target, therefore making derivation of XBD models more efficient and less uncertain.

MC simulation of radiation transport and biochemical processes in microscale timing and spatial dimensions for each of the incoming protons (on the order of 109) is too time consuming, even for supercomputers, given the complexity of quantum physics equations embedded and implemented by MC. However, the full, LET dependent, quantum physics processes can be simplified in complex MC at microscale radiation transport to simulate the FLASH biological effects. Here, an inverse solution to IPO-IMPT that can be implemented on a modest cluster is provided. Such an inverse solution to IPO-IMPT can potentially improve cancer patient outcomes because microscale radiation transport underlies biochemical processes responsible for FLASH sparing of OARs.

The optimization technique described herein is flexible enough that additional optimization parameters and objectives may be easily added. For example, the downstream distance from the nozzle of the patient can have a significant impact on dose rate. In some embodiments, this distance is fixed, but in other embodiments, this value is made variable and included in the optimization. Similarly, other quantities such as material density, beam current, dose threshold for the FLASH effect, dose rate threshold for the FLASH effect, etc., are fixed in some embodiments, and optimized in other embodiments using this technique.

In the limited preclinical feasibility study, the focus was on the reduction of the unoptimized wide spread of dose, XBD (DADR) and XBD (LETd) distributions using IPO-IMPT for a minipig lung to help with quick convergence of XBD model derivations. The preliminary case was chosen to show the capability of IPO-IMPT to inversely solve the most relevant issues for preclinical application, because the lung is considered to have largest benefit from FLASH sparing with the biggest impact for often-fatal ultra-central lung cancer. Inverse solutions to IBO-IMPT for sparing of other organs, such as the esophagus, central airways, and heart, can be derived. FIG. 5 shows that it is feasible for our preliminary SIEMAC to reduce the spreads of dose, dose rate, and LET distribution. Giving researchers control over the average values and spreads of dose, dose rate, and LET distributions can minimize the overlaps of dose, dose rate and LET among irradiations, therefore improving the efficiency with which XBD models can be derived and reducing the number of needed animal irradiations. In some embodiments, alternative methods of quantifying the distribution spreads, besides FWHM and integrated area, may be used. Minimization of overlaps of dose, dose rate and LET among irradiations are vital for biologists to separate their XBD (DADR) and XBD (LET_{d}) from physical dose contribution, which can be badly entangled among these three terms and multiple irradiations without careful optimizations, in observed OAR toxicities.

In addition to XBD, alternative definitions of dose rate besides DADR may prove to be more useful, and could allow more elegant XBD (DADR) and XBD (LET_{d}) models. The definition of tᵢ^{RIG} (equation 7) can also be varied. Associated with spot peak dose rate, RIG can potentially provide solutions more relevant to FLASH biology. Although SIEMAC's current tapping of the underlying quantum physics radiation transport is rudimentary, further accessing the three microscopic dimensions and micro-timing is possible with better computing power and better implementations of inverse optimization. In addition, the objective function can be fine-tuned to account for the varying radiosensitivities of different OARS. For example, different OARS have suggested FLASH dose rate thresholds that vary by more than a factor of 2.

### Example 3: Quality Assurance to Validate that Actual Dose Rates and LETs Correspond to Planned or Predicted Values

### Introduction

Over the past decade, there has been a burgeoning interest in FLASH radiotherapy, where FLASH refers to ultra high dose rates (UHDR), typically above 40 Gy/s. This interest is due to a number of studies that demonstrated that these high dose rates can significantly reduce damage to the healthy tissue of organs at risk (OARs) during treatment when compared to more conventional techniques, without sacrificing tumor killing efficacy. Proton pencil beam scanning (PBS) systems are an especially promising candidate for delivering FLASH treatments since many existing proton therapy centers can be made capable of delivering such beams with minimal overhead. Besides FLASH dose rates, which typically deliver a dose within a few milliseconds, protons offer biological effectiveness related to linear energy transfer (LET) according to their spatial and timing distributions, i.e. quantum physics processes besides the traditional classical physics processes that are described by dose distributions.

One approach to treating patients with a FLASH proton (or other charged particle) beam is to use a patient-specific ridge filter to modulate the beam and therefore deliver a conformal dose distribution within a given beam specific target volume. Advancements in 3D printing technology make fabricating these patient-specific ridge filters accessible and affordable, and 3D printing has already been shown to be a useful tool in radiotherapy applications. However, FLASH treatment planning requires optimization of dose rate and LET in addition to dose, as well as the consideration of the distributions of these three quantities in the OARs, besides the target. Simultaneous optimization of dose and dose rate has been achieved, and simultaneous optimization of dose and LET has also been achieved. Recently, work has been done to show that dose, dose rate, and LET distributions can all simultaneously be optimized using a ridge filter.

All of these new developments have generated an increased demand for innovation in detectors and techniques for measuring dose, dose rate, and LET for UHDR treatment plans. Detectors for measuring dose distributions, such as IBA Dosimetry's DigiPhant+MatriXX PT, have been around for years and continue to be widely used. Dose rate measurements for UHDR irradiations have been demonstrated using a scintillator-based detector in preclinical studies. Hybrid semiconductor pixel detectors based on the Timepix3 chips developed at CERN can be used to measure LET distributions and timing. These detectors have a silicon sensor with 14.08 mm×14.08 mm sensitive regions corresponding to 256×256 pixels (pixel pitch of 55 μm) and nanosecond scale timing resolution. Novel strip ionization chambers have also recently been developed.

The goal of this work is to develop techniques for performing QA measurements for UHDR treatment plans. For example, physical and biological optimization frameworks can be used to generate such plans. The MLSIC and Timepix3-based pixelated silicon detectors, which have never been validated under UHDR conditions without undesirable beam modifications, are selected for measuring dose, dose rate, and LET for a UHDR plan with a ridge filter. Solutions to challenges likely to be encountered with such measurements are also presented.

### Materials and Methods

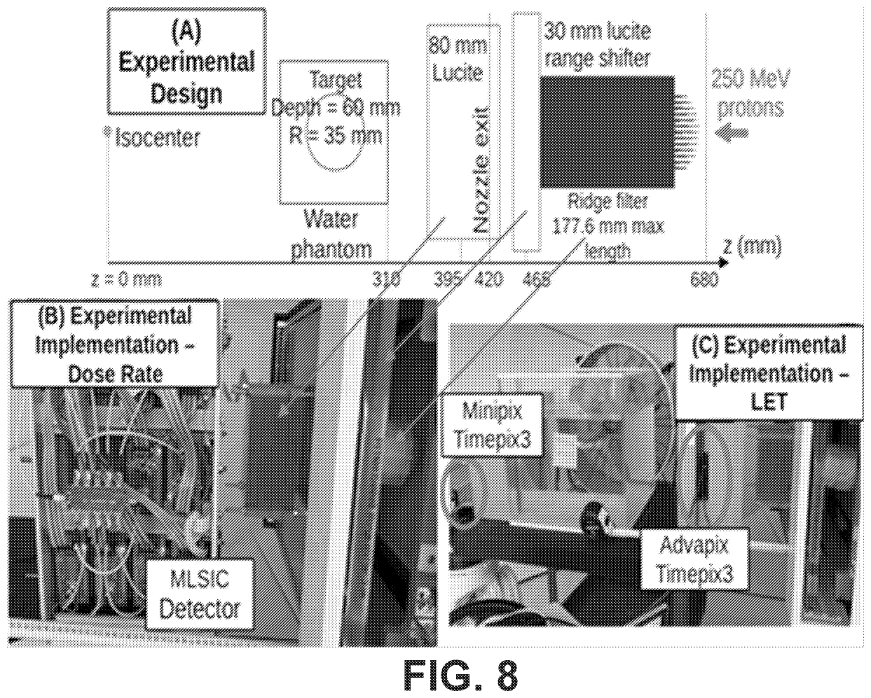

In this work, dose, dose rate, and LET are measured for a simple mock UHDR treatment plan generated using in-house software. The plan included of 250-MeV protons delivered by the Varian ProBeam PBS system according to a circular spot map with 5 mm spacing between each of the 149 spots with uniform intensities. Range shifters, along with a 3D printed ridge filter designed using an algorithm, were used to uniformly irradiate a 70 mm diameter spherical region inside of a water phantom with the target center at a depth of 60 mm, as shown in FIG. 8A. Multiple measurements were performed using different novel detectors and detection techniques in order to get a complete set of dose, dose rate, and LET data in the desired target and target margin locations. The results were validated using a popular commercial detector, IBA Dosimetry's MatriXX PT, as well as by TOPAS Monte Carlo simulations.

Although the treatment plan used was a UHDR plan, we chose to perform some of the dose and LET measurements at low beam currents that did not achieve UHDR. This does not diminish the validity of the measurements, since total dose and LET do not depend on the beam current or the dose rate used. This was done to solve issues with detector saturation and overheating.

To properly design the experiment, perform realistic simulations, and carry out measurements, some preliminary calibration needed to be performed. First, material characterization for the 3D printed ridge filter was conducted using standard techniques. Subsequently, to ensure reliable LET measurements for single particle events, we developed an undersample-and-recover technique which is described below in the Principles of LET Measurements section.

### Experimental Setup

FIG. 8A illustrates the experimental design used in this experiment. A proton beam of 250 MeV energy first impinges upon the ridge filter, followed by traversing an additional 30 mm+80 mm of lucite in order to modulate the protons to achieve the desired depth. Subsequently, the protons deposit their remaining energy in the water phantom. Additionally, the figure contains photographs of the experimental setups for the MLSIC (FIG. 8B) used to simultaneously measure dose and dose rate, and two Timepix3 detectors (FIG. 1C). The Timepix3 detectors were employed together to simultaneously measure LET using the upstream Advapix detector, and timing via prompt gamma rays using the downstream Minipix detector.

The nozzle of the machine is equipped with a laser grid running parallel to the downstream face of the range shifter for safety purposes, such that the beam will be shut off if any of the lasers are blocked. The mounting mechanism of the 80 mm lucite block had to be carefully designed to avoid blocking these lasers. This was done by mounting the block via four narrow bolts that could fit in-between adjacent lasers. The bolts allowed the block to be mounted such that there is a 15 mm air gap between the 30 mm range shifter and 80 mm block, thus avoiding blocking the lasers.

### Principles of LET Measurements

LET is defined as the ratio of the energy a particle deposits along its trajectory (E) to its path length (L) and normalized by the density of the transport medium (Q), i.e.

$$LET = \frac{E}{{L}_{p}} \text{?} \qquad ( 3.1 )$$

$$\text{?} \text{indicates text missing or illegible when filed}$$

Since we use a silicon detector, LET is reported in units of (keV/μm)/(g/cm³) in silicon.

To ensure accurate measurements of LET for single particle events, there are two main parameters that should be accounted for: energy deposition and path length of individual particles through the sensor. Limitations can rise from the imprecision in the calculation of path length when the detector orientation angle is smaller than 30°, where 0° is defined as when the beam is perpendicular to the sensor surface. Moreover, as the Timepix3 chips are measuring the time over threshold (TOT) it is necessary that the acquisition time of each frame to be set high enough so the shaping time would allow registration of all charge produced by a particle. Previously, the lowest frame acquisition time reported was set at 500 μs. However, using the recommended conditions in our setup, a pileup of particles (i.e., saturation) was still noticed. Consequently, we had to employ shorter acquisition times and position the detector perpendicular to the beam, resulting in charge sharing over a smaller area of individual tracks (i.e., fewer pixels) for each detected proton. The shortened acquisition time resulted in an underestimation of the deposited energy due to incomplete charge collection by the detector electronics. Moreover, when the detector was oriented perpendicularly to the beam without making any additional adjustments to the formula used, it led to an underestimate of the LET.

To address these issues, systematic studies of the detector response as a function of both acquisition time and detector angle were conducted. The aim was to derive LET correction factors that could be applied to the collected experimental data. For this purpose, LET distributions were measured with varying frame acquisition times (or detector angles), and then each fitted with a Gaussian to find the peak location. Subsequently, the peak positions were plotted as a function of acquisition time or detector angle. This comprehensive analysis enabled the establishment of correction factors that account for the aforementioned effects and improve the accuracy of our measurements.

Measurements of the LET distributions require some filtering of the data to remove noise and background. A Savitzky-Golay filter was applied to the data to smooth out regions of low statistics. Statistics were limited by the detector overheating when using too high beam current or dose, and by available beam time.

### Measurement Details and Workflow

The proton PBS system used was the Varian ProBeam, which can deliver energies up to 250 MeV at nozzle currents beyond 300 nA with the latest monitor unit (MU) chamber. Each of the 149 spots received 250 MU, where a MU is proportional to the number of protons (Nₚ). The MU to Nₚ conversion factor is energy dependent; at 250 MeV, there are 5.343×10⁶ protons per MU.

Three groups of measurements were performed, one for each of the detector configurations described in FIG. 8, along with a validation run using the MatrixX PT:

The first group of measurements was done using the MLSIC detector. Since the MLSIC is a 4D detector, time-dependent dose and IDR values in 3D space can be collected by running the beam one single time. Two datasets were collected, one with 7 nA nozzle current for a low dose rate measurement, and one with 50 nA nozzle current for a UHDR measurement. The integration duration of the MLSIC of 272 us was used to calculate the average dose rate for each voxel within each time window, i.e. the dose rate for voxel i and time window k is

$$D {R}_{i k} = \frac{{d}_{i k}}{Δ {t}_{k}} = \frac{{d}_{i k}}{272 μ s} \qquad ( 3.2 )$$

wheredᵢₖ is the dose to voxel i during time window k, and Δtₖ is the duration of time window k and is always a constant value of 272 μs. Dose uncertainty for the MLSIC was estimated to be 2.5%.

The MLSIC detector is comprised of x and y strips at different depths and principally reconstructs the 3D dose and dose rate distributions with certain assumptions about the dose profiles of the pencil beams. The presence of the ridge filter causes irregular dose profiles and makes dose reconstruction from MLSIC data challenging, therefore the results for relative dose for just one selected spot are demonstrated, specifically the first spot of the spot map, which lies on the lateral margin.

The second group of measurements was done with two Timepix3 detectors; the primary (upstream Advapix) detector was used to measure LET while the secondary (downstream Minipix) detector was used to measure timing by detecting prompt gamma rays. Several datasets were collected for different depths and lateral offsets of the primary detector. Our minimum nozzle current of 0.8 nA was used to avoid detector saturation. In these measurements, we also lowered the plan MU from 250 MU per spot to 5 MU per spot to avoid overheating the detector.

The third group of measurements was done with a 2D DigiPhant+MatriXX PT detector moving along the depth direction for validation. Dose in the xy-plane was measured at 17 different depths between 28 mm and 95 mm. Dose uncertainty for the MatriXX PT was estimated to be 1%. A nozzle current of 10 nA was used.

### Monte Carlo Simulations

For further validation, TOPAS Monte Carlo simulations were performed of each of the previously described measurements. Following the guidance of AAPM Task Group 268, we have provided Table 1 which summarizes relevant details of the simulation.

TABLE 1
Monte Carlo simulation details with items
recommended by AAPM Task Group 268.

| Item | Description |
|---|---|
| Code version | TOPAS 3.7 |
| Validation | TOPAS for PBS applications has been validated |
| | against measurements in previous works |
| Timing | Each simulation took a few hours on an Intel Xeon |
| | Gold 5120 2.2 GHz 56 thread CPU |
| Source | Emittance source with parameters from Varian |
| | ProBeam commissioning data |
| Physics and | TOPAS default settings |
| transport | |
| Scoring | Dose to medium and dose averaged proton LET |
| Number of | 50k protons per spot for LET, 2M protons per |
| histories | spot for dose |
| Statistical methods | Batch method |
| Postprocessing | Savitzky-Golay filter |

The experimental measurements were compared to the simulations using a gamma analysis that accounts for differences in pixel size, statistical uncertainty, and uncertainty in detector positioning. A 3D gamma analysis was performed for each measurement using in-house software following the algorithm. For LET distributions, experimental data was compared to simulations by calculating the Bhattacharyya distance.

### Results

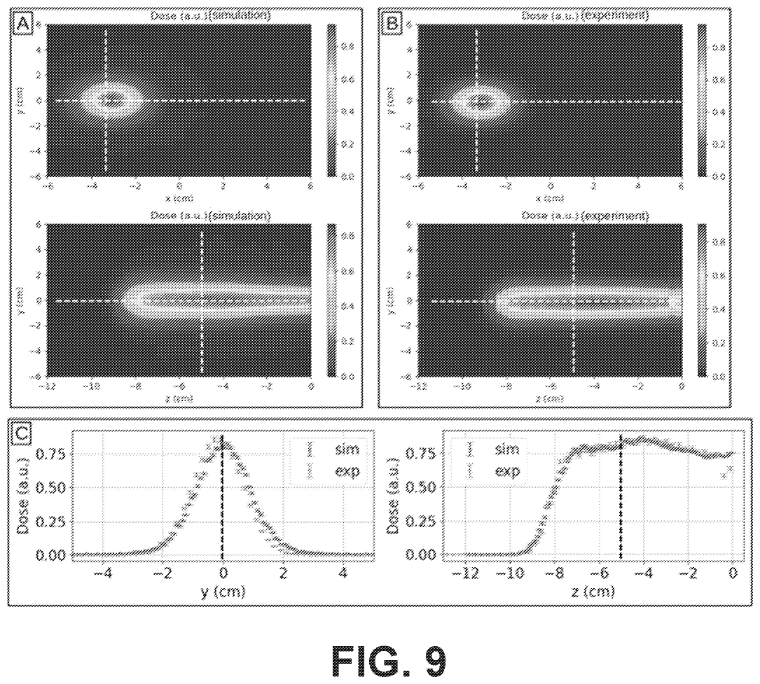

The data for the first spot can be seen in FIG. 9. A 3D gamma analysis was performed using in-house software. Using a standard 3%/3 mm criteria on points with at least 5% of the maximum dose, all 32 measurements had a passing rate above 90% and 29 of the 32 measurements had a passing rate above 95%.

The measurements done with the MLSIC detector used 7 nA and 50 nA beams each with 149 spots and 250 MU per spot. For the 7 nA beam, the irradiation time (IRT) for the first spot measured by MLSIC was 26.928 ms. This should be compared to the value from Varian log files, which recorded an IRT of 27.265 ms, a 1.2% difference. For the 50 nA beam, the IRT for the first spot measured by MLSIC was 11.832 ms and the log files recorded 11.691 ms for a difference of 1.2%.

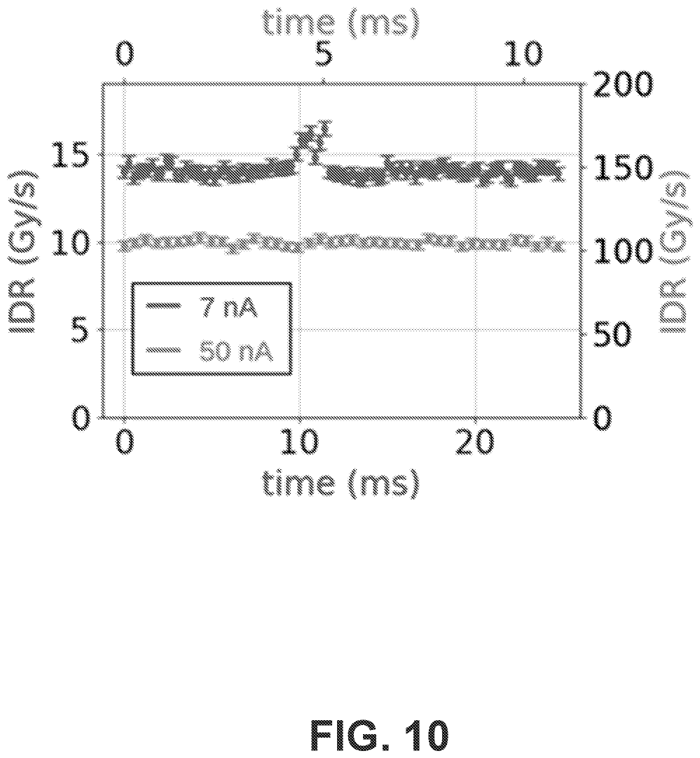

The time-dependent instantaneous dose rate curve for the first spot was also measured. The integration duration of the MLSIC is 272 μs, which corresponds to 99 samples within the time window of the first spot for the 7 nA beam and 44 samples for the 50 nA beam. The relative dose per sample, which remained fairly constant but for fluctuations on the order of 10% or less, was scaled to absolute dose using simulation data. Results for the first spot are plotted in FIG. 10 at a depth of 50 mm along the central axis of the spot. The small variations in dose rate are most likely due to fluctuations in the anode, cathode, and RF of the cyclotron.

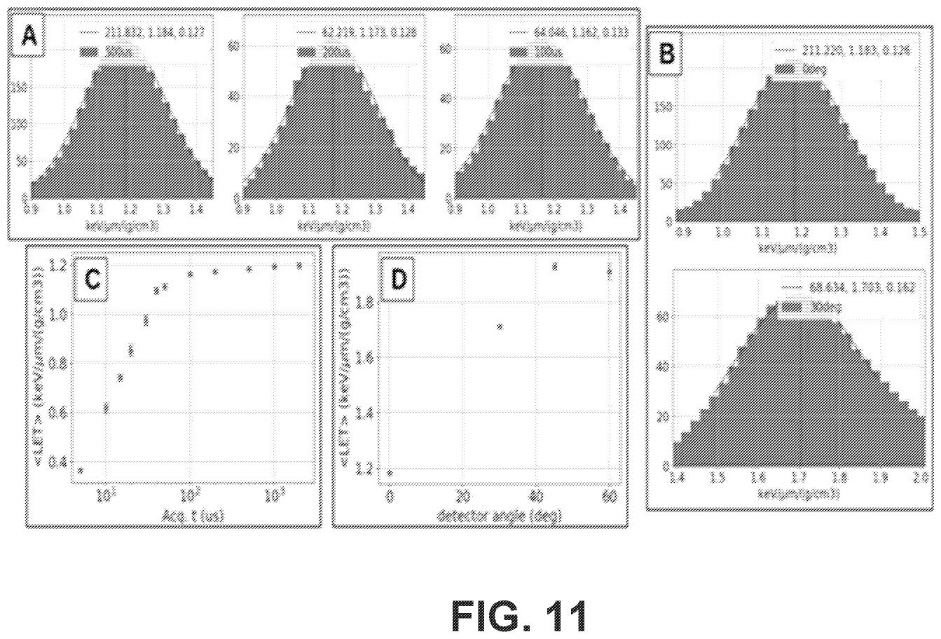

For the LET correction factors, each LET distribution was fit with a Gaussian in order to find the location of the peak, as shown in FIGS. 11A and 11B. One standard deviation of the fit parameter estimates is used as error bars. The peak positions were then plotted as a function of acquisition time (FIG. 11C) and detector angle (FIG. 11D) which can then be used to calculate the necessary LET correction factors.

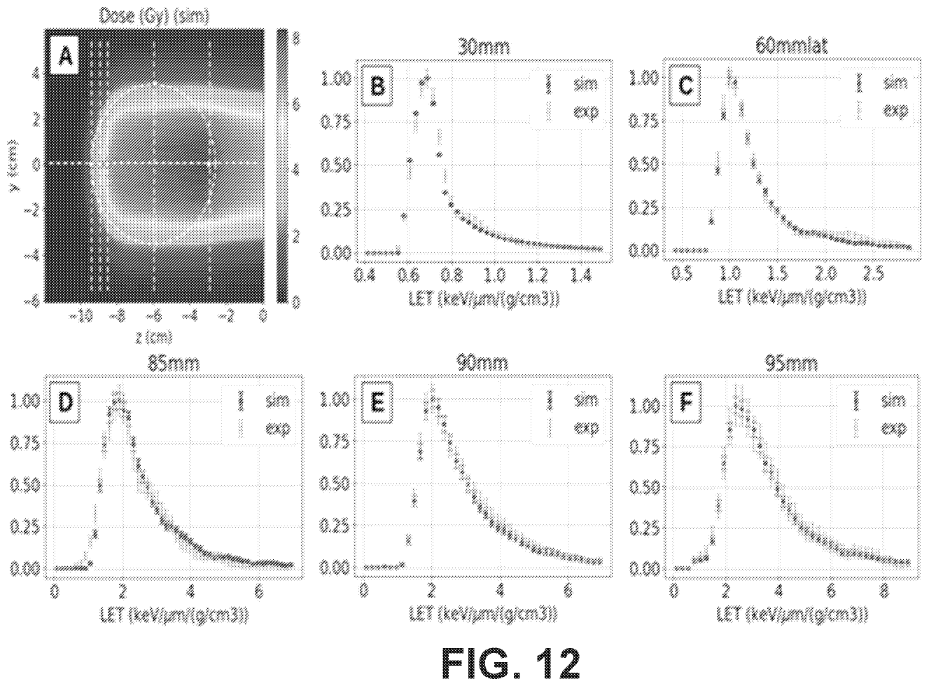

FIG. 12 shows the corrected experimental and simulated LET distributions for five locations, represented by small circles in panel A: 30 mm depth on axis (panel B), 60 mm depth at lateral margin (panel C), 85 mm depth on axis (panel D), 90 mm depth on axis (panel E), and 95 mm depth on axis (panel F). The similarity between each experimental and simulated result was quantified by calculating the Bhattacharyya distance, which was 3.3e-3, 2.5e-3, 1.3e-2, 4.4e-4, 6.7e-4 for FIG. 12 panels B-F, respectively. The data show more and more high LET (greater than 4 keV/μm) components with increasing depth.

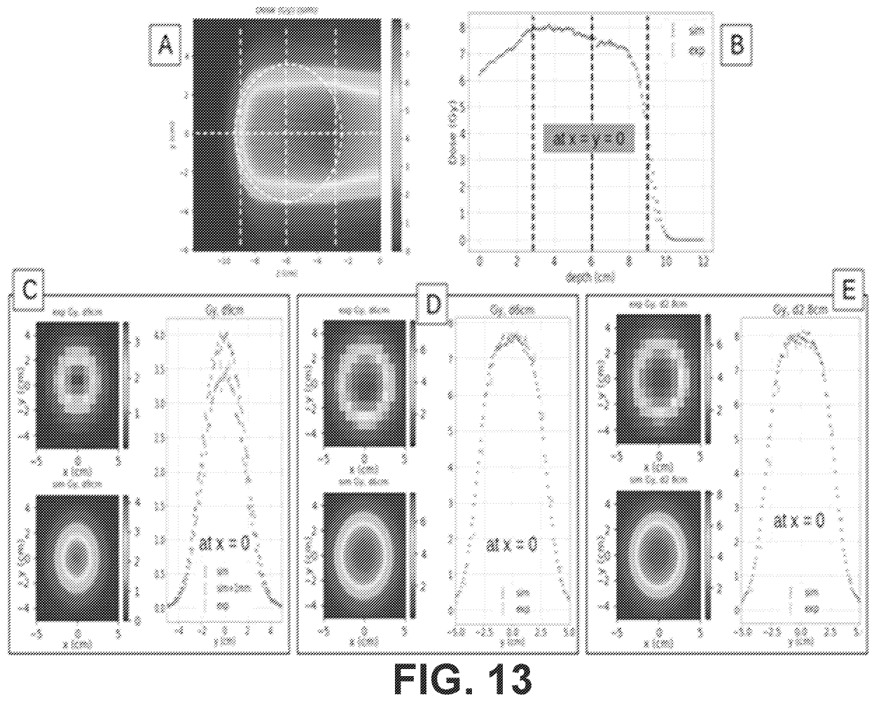

FIG. 13 shows the absolute dose measured with the MatriXX PT at 10 nA along with simulations for comparison. In FIG. 13C, which shows data at depth 90 mm, which is within the distal falloff region, an additional simulation result at depth 91 mm is shown to demonstrate that the 10% disagreement between data and simulation represents a less than 1 mm difference. A gamma analysis using in-house software was performed between the dose measured with the MatriXX PT and TOPAS simulation that accounts for differences in pixel size (1 mm for simulation, 7.619 mm for experiment), statistical uncertainty, and uncertainty in detector positioning. The gamma analysis was limited to points greater than 5% the maximum dose of each measurement. Using a standard 3%/3 mm criteria, 16 of the 17 measurements had a passing rate greater than 90% (representing depths from 28 mm to 94 mm). The measurement at a depth of 95 mm was the only measurement with a gamma passing rate less than 90%, though the maximum dose in this measurement was 0.751 Gy, representing a dose less than 10% the maximum dose of the plan. Of the 16 measurements which had at least a 90% passing rate, 15 passed with at least 95% of points meeting the 3%/3 mm criteria (representing depths from 28 mm to 94 mm).

### Discussion

There have been several optimization methods to include FLASH dose rate and/or LET. Here time-dependent instant dose rate curves and LET spectra are used to describe the quantum physics processes underlying such integrated optimizations. This example represents the first validation of quantum physics key parameters under unmodified primary FLASH beams. This is possible due to the novel under-sample and recover method using microsecond acquisition time to avoid saturation and recover the original LET values by calibration of under response of such short acquisitions.

Overall, the measurements agreed very well with simulations. Small offsets of ˜1 mm caused by imperfect detector positioning can be seen in FIGS. 9C, 13D, and 13E. FIG. 13C shows another <1 mm effect caused by imperfect modeling of the distal falloff region, which is known to be challenging. These small effects were accounted for in the gamma analysis. For the LET results, the data and simulations typically agreed within statistical error bars. In a small number of bins, the disagreement is slightly larger, and can be explained by incomplete filtering of noise and background.

As FLASH radiotherapy continues to grow in popularity, advancements in detector technology will be important. The measurements with the MLSIC detector demonstrate a proof of concept for measuring the 4D dose distribution at UHDR. This work also represents a proof of concept for conducting preclinical FLASH trials, as the experiments were run with modifications to the nozzle in a non-invasive way without tripping any safety mechanisms.

In FIG. 9, shown is a classical physics representation of a spot dose distribution. This concept of a spot is invalid under FLASH, which in reality is represented by quantum physics uncertainty of positioning, which can be seen over time by FIG. 10 with uncertainty of proton energy represented by LET spectra in FIG. 12.

Although this work was done with a ridge filter and proton beam, the methods described have broad applicability to non-ridge and non-proton therapy modalities as well. For example, these methods could apply to other energetic particle beams. Furthermore, with the feasibility of simultaneously optimizing dose, dose rate, and LET, these techniques will be important for validating solutions to that optimization problem, as it is necessary to validate the underlying quantum physics processes related to FLASH dose rate and LET.

The quantum processes of how incoming protons deposit dose over milliseconds (FLASH IDR) and how proton energies are distributed over microns (LET) both contribute to radiobiological effectiveness (RBE). In OAR, RBE sparing provided by FLASH can be negated by RBE enhancement as a result of high LET. Therefore, explicitly tapping into the quantum physics parameters (LET and dose rate) besides classical physics parameter (dose) can provide multiple and better solutions of the integrated optimization of dose, dose rate and LET.

### Supplemental Information for Example 3

The ridge filter was 3D printed using a proprietary resin from Formlabs called Rigid 4000. Since the exact chemical formula of the resin is not public information, we had to develop a technique for realistically characterizing and modeling the material for simulations. To do this, an 80 mm long and 20 mm diameter cylinder was printed with the same resin. We then used a proton beam and commercial Zebra detector from IBA to measure the R80 of the protons with and without the cylinder in the beam. The water equivalent thickness (WET) of the cylinder is then

$$WET = R {80}_{without} - R {80}_{with} . \qquad ( 3.3 )$$

From here, the relative stopping power (RSP) can be calculated by

$$RSP = \frac{WET}{material length} . \qquad ( 3.4 )$$

To realistically model the stopping power of this material in the simulations, we assumed a density [symbol] ᵣₑₛᵢₙ=(RSP) [symbol] _{water} To realistically model the scattering power, we assumed a chemical composition of xH₂O+(1−x)SiO₂, and then simulated many values of x between 0 and 1 and compared the dose profile to data to choose the best value for x. SiO₂ was chosen because it is known to be one of the major ingredients in resins besides PMMA.

The characterization of the resin yielded an RSP value of 1.265 and an x-value of 0.95.

Dose rate is calculated by simply dividing the total dose by the IRT. Since the dose measurements have already been completed as described above, dose rate measurements can be simplified to just measuring the IRT.

In this work, we tested the feasibility of simultaneously measuring LET and IRT with a two-detector setup. Due to challenges with measuring LET described above, these measurements were done at low current (i.e. not UHDR).

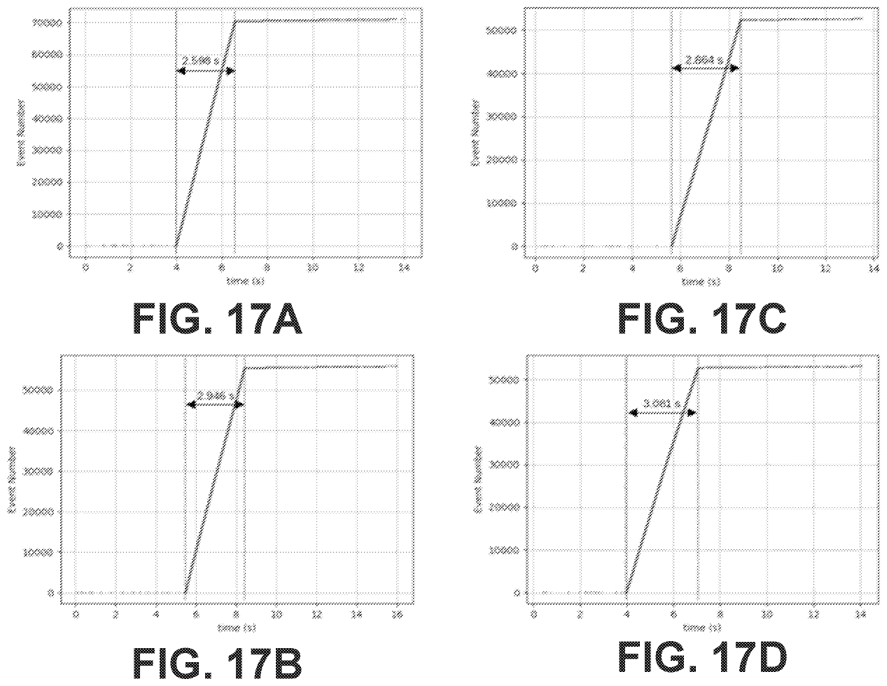

FIG. 17 shows timing measured with Minipix Timepix3 at 250 MeV, 10 nA, 250 MU per spot, and 149 spots. Four repetitions were done to demonstrate reproducibility. The plots show clearly when the beam first turns on and when it stops, making extraction of the timing information from the data very straightforward, in this case between 2.598 and 3.081 seconds. Varian log files recorded IRTs ranging from 2.58553 to 3.06717 seconds, with an average of 2.86393 seconds, showing good agreement with the detector within 0.3%.

Commissioning data from Raystation reports that at 250 MeV, there are 5.3×10⁶ protons per MU. Therefore, the expected IRT for these measurements is

$$\frac{( 5.3 \times {10}^{6} prot / MU ) \times ( 250 MU / spot ) \times ( 149 spots ) \times ( 1.6 \times {10}^{- 19} C / prot )}{( 10 \times {10}^{9} C / second )} \qquad ( 3.5 )$$

or 3.159 seconds. The measured time values are 2-18% lower than this calculated value, this is most likely due to the cyclotron to nozzle conversion efficiency on the day of the measurement being lower than the nominal value.

The four timing results from FIG. 17 show fluctuations of 7-9% from the average. This is not unexpected due to fluctuations in the cyclotron to nozzle conversion efficiency.

The terminology used herein is for purposes of describing particular embodiments only and is not intended to be limiting. The defined terms are in addition to the technical, scientific, or ordinary meanings of the defined terms as commonly understood and accepted in the relevant context.

The terms “a,” “an” and “the” include both singular and plural referents, unless the context clearly dictates otherwise. Thus, for example, “a device” includes one device and plural devices. The terms “substantial” or “substantially” mean to within acceptable limits or degrees acceptable to those of skill in the art. For example, the term “substantially parallel to” means that a structure or device may not be made perfectly parallel to some other structure or device due to tolerances or imperfections in the process by which the structures or devices are made. The term “approximately” means to within an acceptable limit or amount to one of ordinary skill in the art. Relative terms, such as “over,” “above,” “below,” “top,” “bottom,” “upper” and “lower” may be used to describe the various elements' relationships to one another, as illustrated in the accompanying drawings. These relative terms are intended to encompass different orientations of the device and/or elements in addition to the orientation depicted in the drawings. For example, if the device were inverted with respect to the view in the drawings, an element described as “above” another element, for example, would now be below that element.

Relative terms may be used to describe the various elements' relationships to one another, as illustrated in the accompanying drawings. These relative terms are intended to encompass different orientations of the device and/or elements in addition to the orientation depicted in the drawings.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

1. A radiation therapy method, comprising:
receiving a beam of particles;
directing the beam of particles to a patient specific sparse passive filter to form an adjusted beam of particles, wherein the patient specific sparse passive filter is configured to modulate the beam of particles, wherein the patient specific sparse passive filter is formed based on a simultaneous optimization of a dose of particles from the beam of particles, a dose-averaged dose rate (DADR) of particles from the beam of particles, and dose-averaged linear energy transfer (LET_{d}) of the particles from the beam of particles to target a target area of a patient and substantially spare organs at risk (OARs); and
administering the adjusted beam of particles to the target area of the patient.

2. The radiation therapy method of claim 1, wherein the particles are high energy charged particles, optionally wherein the high energy charged particles are electrons, protons, or heavy ions.

3. The radiation therapy method of claim 1, wherein the particles are protons.

4. The radiation therapy method of claim 1, wherein the radiation therapy method is proton FLASH radiotherapy.

5. (canceled)

6. A system for radiation therapy, comprising:
a particle source for a beam of particles; and a patient specific sparse passive filter, wherein the patient specific sparse passive filter is configured in the system to receive the beam of particles, wherein the patient specific sparse passive filter is configured to modify the beam of particles to form an adjusted beam of particles, wherein the patient specific sparse passive filter is formed based on a simultaneous optimization of a dose of particles from the beam of particles, a dose-averaged dose rate (DADR) of particles from the beam of particles, and dose-averaged linear energy transfer (LET_{d}) of the particles from the beam of particles to target a target area of a patient and substantially spare organs at risk (OARs).

7. The system for radiation therapy of claim 6, wherein the particles are high energy charged particles, optionally wherein the high energy charged particles are electrons, protons, or heavier ions than protons.

8. The system for radiation therapy of claim 6, wherein the particles are protons.

9. The system for radiation therapy of claim 6, wherein the radiation therapy method is proton FLASH radiotherapy.

10. The system for radiation therapy of claim 6, wherein components of the patient specific sparse passive filter are partially or entirely recessed within a nozzle adjacent a patient.

11. The system for radiation therapy of claim 10, wherein positioning the patient specific sparse passive filter recessed within the nozzle Increase the dose rate about 40% or more as compared to the patient specific sparse passive filter positioned outside of the nozzle.

12-19. (canceled)

20. A patient-specific sparse passive filter for simultaneous intensity and energy modulation in proton therapy, the patient-specific sparse passive filter designed by the process of:
determining an initial geometry of a sparse passive filter based at least in part on a scan of a patient;
determining a dose influence matrix and an LET influence matrix;
simulating a plurality of geometry variations using a particle simulation; and
optimizing output data from the particle simulation to determine an optimized geometry; the optimization being based at least in part on the dose influence matrix and the LET influence matrix.

21. The patient-specific sparse passive filter of claim 20, wherein the process further comprising receiving the scan of a patient.

22. The patient-specific sparse passive filter of claim 20, wherein the process further comprising applying a ray tracing algorithm to the scan of the patient, and determining the initial geometry based at least in part on a result of the ray tracing algorithm.

23. The patient-specific sparse passive filter of claim 20, wherein the process further comprising optimizing a dose of protons from the beam of protons, a dose-averaged dose rate (DADR) of protons from the beam of protons, and dose-averaged linear energy transfer (LET_{d}) of the protons from a beam of protons from the output data from the particle simulation.

24. The patient-specific sparse passive filter of claim 20, wherein the process for determining a dose influence matrix and an LET influence matrix and simulating a plurality of geometry variations using a particle simulation are accomplished in parallel.

25. The patient-specific sparse passive filter of claim 20, wherein the process further comprising fabricating the patient-specific sparse passive filter based at least in part on the optimized geometry.

26-37. (canceled)
